(19) United States
(12) Patent Application Publication
(10) Pub. No.: US 2024/0317742 A1
(43) Pub. Date: Sep. 26, 2024

(54) METHODS OF PREPARING INTERMEDIATES OF HETEROARYL-KETONE FUSED AZADECALIN GLUCOCORTICOID RECEPTOR MODULATORS

(71) Applicant: Corcept Therapeutics Incorporated, Menlo Park, CA (US)

(72) Inventors: Hazel Joan Hunt, West Sussex (GB); Gary Patrick Reid, Menlo Park, CA (US); Jeffrey Mark Dener, Menlo Park, CA (US); Adam Daisuke Gammack Yamagata, Enfield (GB); Alexander Fergus Williams, Hertford (GB); Liam Joseph Badiola, Hoddesdon (GB); Evangeline Rose Newby, Nottinghamshire (GB)

(21) Appl. No.: 18/584,163

(22) Filed: Feb. 22, 2024

Related U.S. Application Data
(60) Provisional application No. 63/486,753, filed on Feb. 24, 2023.

(51) Int. Cl. C07D 471/04; C07D 217/26

(52) U.S. Cl. CPC C07D 471/04; C07D 217/26

(57) ABSTRACT

The present invention provides methods of preparing intermediates of heteroaryl-ketone fused azadecalin glucocorticoid receptor modulators, and compositions having low impurity levels.

### CROSS-REFERENCES TO RELATED APPLICATIONS

This application claims priority to U.S. Provisional Application No. 63/486,753, filed Feb. 24, 2023, which is incorporated by reference herein in its entirety for all purposes.

### BACKGROUND

There are two types of high-affinity receptors for corticosteroids; the type I (mineralocorticoid receptor, MR) and the type II (glucocorticoid receptor (GR), or cortisol receptor, GR). In most species, including man, the physiological glucocorticoid is cortisol (hydrocortisone). Glucocorticoids are secreted in response to ACTH (corticotropin), which shows both circadian rhythm variation and elevations in response to stress and food. Cortisol levels are responsive within minutes to many physical and psychological stresses, including trauma, surgery, exercise, anxiety and depression. Cortisol is a steroid and acts by binding to an intracellular, glucocorticoid receptor (GR). In man, glucocorticoid receptors are present in two forms: a ligand-binding GR-alpha of 777 amino acids; and, a GR-beta isoform which lacks the 50 carboxy terminal residues. Since these include the ligand binding domain, GR-beta is unable to bind ligand, is constitutively localized in the nucleus, and is transcriptionally inactive.

The biologic effects of cortisol, including those caused by hypercortisolemia, can be modulated at the GR level using receptor modulators, such as agonists, partial agonists and antagonists. Several different classes of agents are able to block the physiologic effects of GR-agonist binding. These antagonists include compositions which, by binding to GR, inhibit the ability of an agonist to effectively bind to and/or activate the GR. One such known GR antagonist, mifepristone, has been found to be an effective anti-glucocorticoid agent in humans (Bertagna (1984) J. Clin. Endocrinol. Metab. 59:25). Mifepristone binds to the GR with high affinity, with a dissociation constant (K_{d}) of 10⁻⁹M (Cadepond (1997) Annu. Rev. Med. 48:129). Additional glucocorticoid receptor modulator compounds include dazucorilant (CORT113176) and relacorilant (CORT125134) described previously in PCT Publication No. WO 2013/177559 and U.S. Pat. No. 8,859,774, exicorilant (CORT125281) and zavacorilant (CORT125329) described previously in PCT Publication No. WO2015/077530 and U.S. Pat. No. 10,047,082 and CORT108297 described previously in PCT Publication No. WO2010/132445 and U.S. Pat. No. 8,889,867. What is needed in the art are new methods of preparing intermediates of GR receptor modulators having higher purity. Surprisingly, the present invention meets these and other needs.

### BRIEF SUMMARY OF THE INVENTION

In one embodiment, the present invention provides a method of preparing a compound of Formula I:

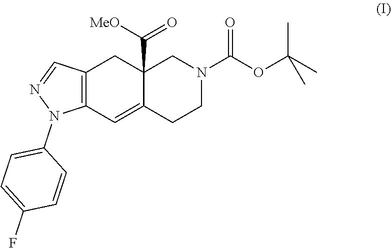 [SMILES: COC(=O)[C@]12Cc3cnn(-c4ccc(F)cc4)c3C=C1CCN(C(=O)OC(C)(C)C)C2]

comprising: (a) forming a first reaction mixture comprising a compound of Formula II:

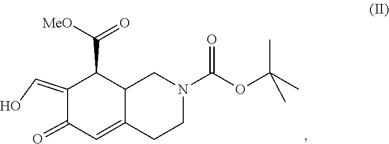 [SMILES: COC(=O)[C@@H]1/C(=C/O)C(=O)C=C2CCN(C(=O)OC(C)(C)C)CC21]

Mg(OAc)₂, and 4-fluorophenylhydrazine HCl:

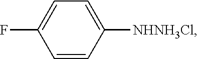

under conditions suitable to prepare the compound of Formula I in a yield of at least 80% and a purity of at least 98%.

In another embodiment, the present invention provides a method of preparing a compound of Formula II:

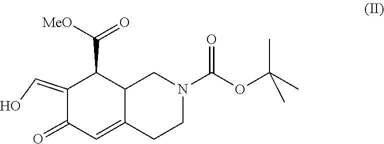 [SMILES: COC(=O)[C@@H]1/C(=C/O)C(=O)C=C2CCN(C(=O)OC(C)(C)C)CC21]

the method comprising: (c) forming a third reaction mixture comprising an alkylformate, a non-nucleophilic base, and a compound of Formula III:

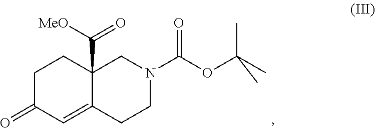 [SMILES: COC(=O)[C@]12CCC(=O)C=C1CCN(C(=O)OC(C)(C)C)C2]

thereby preparing the compound of Formula II.

In another embodiment, the present invention provides a composition comprising: a compound of Formula I in an amount of at least 99%:

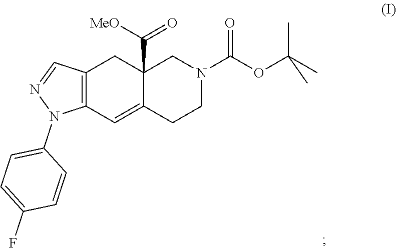 [SMILES: COC(=O)[C@]12Cc3cnn(-c4ccc(F)cc4)c3C=C1CCN(C(=O)OC(C)(C)C)C2]

and
one or more impurity in an amount of from 0.01 to 1%.

### DETAILED DESCRIPTION OF THE INVENTION

### I. GENERAL

The instant disclosure describes new methods of preparing the intermediate compound of Formula I, 6-(tert-butyl) 4a-methyl (R)-1-(4-fluorophenyl)-1,4,7,8-tetrahydro-6H-pyrazolo[3,4-g]isoquinoline-4a,6(5H)-dicarboxylate, having lower impurity levels than the methods previously described. The new methods of preparing the compound of Formula I have improved process safety and cost effectiveness, and can be prepared at larger scale, compared to known methods. The compound of Formula I can be prepared as in Example 33 of U.S. Pat. No. 7,928,237.

### II. DEFINITIONS

“About” when referring to a value includes the stated value +/−10% of the stated value. For example, about 50% includes a range of from 45% to 55%, while about 20 molar equivalents includes a range of from 18 to 22 molar equivalents. Accordingly, when referring to a range, “about” refers to each of the stated values +/−10% of the stated value of each end of the range. For instance, a ratio of from about 1 to about 3 (weight/weight) includes a range of from 0.9 to 3.3.

“Forming a reaction mixture” refers to the process of bringing into contact at least two distinct species such that they mix together and can react. It should be appreciated, however, the resulting reaction product can be produced directly from a reaction between the added reagents or from an intermediate from one or more of the added reagents which can be produced in the reaction mixture.

“Dissolve”, “dissolving” or “dissolution” refers to a solid material that is substantially soluble in a particular solvent. For example, the solid material can be greater than 90% soluble in the solvent, or greater than 91, 92, 93, 94, 95, 96, 97, 98, or greater than 99% soluble in the solvent.

“Substantially free” refers to a composition having an undesired component in an amount less than 5%, less than 1%, less than 0.5% or even less than 0.1% by weight percentage (w/w) or HPLC peak area.

“Aqueous phase” or “aqueous mixture” refers to a mixture containing water and other water-miscible solvents capable of dissolving water-soluble organic and inorganic compounds. The aqueous phase is substantially immiscible with the organic phase.

“Organic phase” refers to a mixture containing water-miscible or-immiscible solvents capable of dissolving either or both of water-soluble and water-insoluble organic compounds. The organic phase of the present invention can be formed from one or more organic solvents. Exemplary organic solvents can be non-polar aprotic solvents, polar aprotic solvents, and polar protic solvents. Representative solvents include, but are not limited to, pentanes, hexanes, hexane, heptane, benzene, toluene, cyclopentyl methylether (CPME), diethyl ether, methyl t-butylether (MTBE), tetrahydrofuran (THF), 2-methyltetrahydrofuran (2-MeTHF), acetone, methyl ethyl ketone, N,N-dimethylacetamide (DMA/DMAc), N-methylpyrrolidinone (NMP), ethyl acetate, isopropyl acetate (iPrOAc), acetonitrile, methylene chloride, chloroform, etc. The organic phase is substantially immiscible with the aqueous phase.

“Separating,” or “separation” refers to one or more processes to physically isolate one or more components of a mixture, with the primary objective of removing one or more undesired components (byproducts, side-products, contaminants and/or impurities) present in the mixture to afford the desired component or product which is substantially free of the undesired components.

“Acid” refers to a compound that is capable of donating a proton (H⁺) under the Bronsted-Lowry definition, or is an electron pair acceptor under the Lewis definition. Acids useful in the present invention are Bronsted-Lowry acids that include, but are not limited to, alkanoic acids or carboxylic acids [formic acid, acetic acid, citric acid, lactic acid, oxalic acid, trifluoroacetic acid (TFA), etc.], sulfonic acids and mineral acids, as defined herein. Mineral acids are inorganic acids such as hydrogen halides [hydrofluoric acid (HF), hydrochloric acid (HCl), hydrobromic acid (HBr), etc.], halogen oxoacids (hypochlorous acid, perchloric acid, etc.), as well as sulfuric acid (H₂SO₄), nitric acid (HNO₃), phosphoric acid (H₃PO₄), chromic acid and boric acid. Sulfonic acids include methanesulfonic acid (CH₃SO₃H; MSA), benzenesulfonic acid (C₆H₅SO₃H), p-toluenesulfonic acid (4-CH₃C₆H₄SO₃H; pTsOH), trifluoromethanesulfonic acid (CF₃SO₃H; TfOH), camphorsulfonic acid, among others.

“Non-nucleophilic base” refers to a base that is a moderate to strong base but at the same time is a poor nucleophile. Representative non-nucleophilic bases include bases such as potassium carbonate, sodium carbonate, alkoxides such as potassium tert-butoxide and sodium tert-butoxide, hexamethylsilazane (HMDS), lithium hexamethyldisilazane, sodium hexamethyldisilazine, potassium hexamethyldisilazane, lithium diisopropylamine (LDA), lithium hydride, sodium hydride, potassium hydride, n-butyl lithium, as well as amine bases, such as triethylamine (Et₃N), N,N-diisopropylethylamine (iPr₂NEt; DIPEA), 1,8-diazabicycloundec-7-ene (DBU), 1,5-diazabicyclo(4.3.0)non-5-ene (DBN), 1,4-diazabicyclo[2.2.2]octane (DABCO), N,N-diethylaniline, pyridine, 2,6-lutidine, 2,4,6-collidine, 4-dimethylaminopyridine, and quinuclidine. Non-nucleophilic base includes non-nucleophilic amine bases.

“Solvent” refers to a substance, such as a liquid, capable of dissolving a solute. Solvents can be polar or non-polar, protic or aprotic. Polar solvents typically have a dielectric constant greater than about 5 or a dipole moment above about 1.0, and non-polar solvents have a dielectric constant below about 5 or a dipole moment below about 1.0. Protic solvents are characterized by having a proton available for removal, such as by having a hydroxy or carboxy group. Aprotic solvents lack such a group. Representative polar protic solvents include alcohols (methanol, ethanol, propanol, isopropanol, etc.), acids (formic acid, acetic acid, etc.) and water. Representative polar aprotic solvents include dichloromethane, chloroform, tetrahydrofuran, diethyl ether, 1,4-dioxane, acetone, ethyl acetate, dimethylformamide, dimethylacetamide, acetonitrile and dimethyl sulfoxide. Representative non-polar solvents include alkanes (pentanes, hexanes, etc.), cycloalkanes (cyclopentane, cyclohexane, etc.), benzene, and toluene. Other solvents are useful in the present invention.

“Anti-solvent” refers to a solvent, or solvent system, in which the compound, composition, or mixture, has low solubility. The anti-solvent can be any suitable solvent as described above.

“Heating” refers to raising the temperature of a mixture above room temperature, or from a lower temperature to a higher temperature.

“Cooling” refers to lowering the temperature of a mixture below room temperature, or from a higher temperature to a lower temperature.

“Room temperature” is the range of air temperatures generally considered to be suitable for human occupancy, or between about 15 degrees Celsius (59 degrees Fahrenheit) and 25 degrees Celsius (77 degrees Fahrenheit).

“Vacuum” or “reduced pressure” refers to a pressure that is less than atmospheric pressure. Atmospheric pressure is measured as about 1013 mbar, 760 mm Hg, or about 14.7 psi. Accordingly, vacuum can be less than 1013 mbar, or less than 100, 10, 1, 0.1, or less than 0.01 mbar.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Alkylformate” refers to a compound of the formulat HC(O)OR, where R is an alkyl group. Representative alkylformates include methylformate, HC(O)OMe; ethylformate, HC(O)OEt; nbutylformate, HC(O)OCH₂CH₂CH₂CH₃; and isoamylformate, HC(O)OCH₂CH₂CH(CH₃)₂.

“Alkoxide” refers to the anion ⁻OR, where R is an alkyl group. Representative alkoxides include, but are not limited to, methoxide, ethoxide, iso-propoxide, and t-butoxide.

“Composition” as used herein is intended to encompass a product comprising the specified ingredients in the specified amounts, as well as any product, which results, directly or indirectly, from combination of the specified ingredients in the specified amounts. By “pharmaceutically acceptable” it is meant the carrier(s), diluent(s) or excipient(s) must be compatible with the other ingredients of the formulation and not deleterious to the recipient thereof.

### III. METHOD OF PREPARING FORMULA I

The compound of Formula I, 6-(tert-butyl) 4a-methyl (R)-1-(4-fluorophenyl)-1,4,7,8-tetrahydro-6H-pyrazolo[3,4-g]isoquinoline-4a,6(5H)-dicarboxylate:

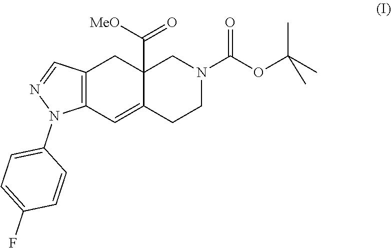 [SMILES: COC(=O)C12Cc3cnn(-c4ccc(F)cc4)c3C=C1CCN(C(=O)OC(C)(C)C)C2]

can be prepared as described in Example 33 of U.S. Pat. No. 7,928,237. In some embodiments, the present invention provides a method of preparing a compound of Formula I:

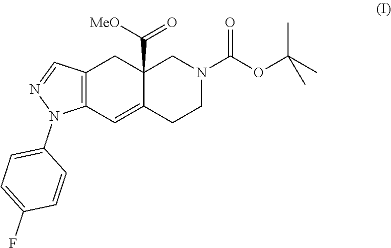 [SMILES: COC(=O)[C@]12Cc3cnn(-c4ccc(F)cc4)c3C=C1CCN(C(=O)OC(C)(C)C)C2]

comprising: (a) forming a first reaction mixture comprising a compound of Formula II:

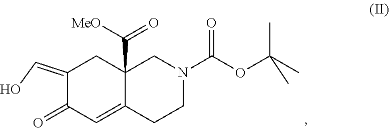 [SMILES: COC(=O)[C@]12C/C(=C/O)C(=O)C=C1CCN(C(=O)OC(C)(C)C)C2]

Mg(OAc)₂, and 4-fluorophenylhydrazine HCl:

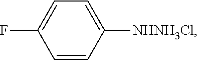

under conditions suitable to prepare the compound of Formula I in a yield of at least 80% and a purity of at least 98%.

Conditions suitable to prepare the compound of Formula I can include any suitable time and temperature, as described below in the examples section.

The Mg(OAc)₂ can be any suitable hydrate. For example, the Mg(OAc)₂ can be a monohydrate, dihydrate, trihydrate, or tetrahydrate. In some embodiments, the method of preparing the compound of Formula I includes the method wherein the Mg(OAc)₂ is Mg(OAc)₂·4H₂O.

The Mg(OAc)₂ can be present in any suitable amount to the compound of Formula II. For example, the Mg(OAc)₂ can be present in an amount of from 0.1 to 5 molar equivalents to the compound of Formula II, or from 0.1 to 4, from 0.1 to 3, from 0.1 to 2, from 0.1 to 1.5, from 0.1 to 1.0, from 0.2 to 1.0, from 0.3 to 0.9, from 0.4 to 0.8, or from 0.5 to 0.7 molar equivalents to the compound of Formula II. Representative amounts of the Mg(OAc)₂ include, but are not limited to, about 0.1 molar equivalents to the compound of Formula II, or about 0.2, 0.25, 0.3, 0.4, 0.5, 0.6, 0.7, 0.75, 0.8, 0.9, 1.0, 1.1, 1.2, 1.3, 1.4, 1.5, 2.0, 2.5, or 3.0 molar equivalents to the compound of Formula II.

In some embodiments, the method of preparing the compound of Formula I includes the method wherein the Mg(OAc)₂ is present in an amount of from 0.5 to 1.0 molar eq. to the compound of Formula II. In some embodiments, the method of preparing the compound of Formula I includes the method wherein the Mg(OAc)₂ is present in an amount of about 0.6 molar eq. to the compound of Formula II.

In some embodiments, the method of preparing the compound of Formula I includes the method wherein the first reaction mixture further includes a first solvent. Any suitable solvent can be used as the first solvent in the first reaction mixture. The first solvent can include, but is not limited to, pentanes, hexanes, heptane, benzene, toluene, diethyl ether, tetrahydrofuran, acetone, ethyl acetate, acetonitrile, methylene chloride, and chloroform. In some embodiments, the method of preparing the compound of Formula I includes the method wherein the first solvent includes pentanes, hexanes, heptane, benzene, or toluene. In some embodiments, the method of preparing the compound of Formula I includes the method wherein the first solvent includes benzene, or toluene. In some embodiments, the method of preparing the compound of Formula I includes the method wherein the first reaction mixture further includes toluene.

The compound of Formula I can be prepared in any suitable yield. For example, the compound of Formula I can be prepared in a yield of at least 10, 20, 30, 40, 50, 60, 65, 70, 75, 80, 85, 90, or at least 95%. In some embodiments, the method of preparing the compound of Formula I includes the method wherein the compound of Formula I can be prepared in a yield of at least 80%.

The compound of Formula I can be prepared in any suitable purity. For example, the compound of Formula I can be prepared in a purity of at least 90%, or 91, 92, 93, 94, 95, 96, 97, 98, or at least 99%. In some embodiments, the method of preparing the compound of Formula I includes the method wherein the compound of Formula I can be prepared in a purity of at least 96%. In some embodiments, the method of preparing the compound of Formula I includes the method wherein the compound of Formula I can be prepared in a purity of at least 97%. In some embodiments, the method of preparing the compound of Formula I includes the method wherein the compound of Formula I can be prepared in a purity of at least 98%. In some embodiments, the method of preparing the compound of Formula I includes the method wherein the compound of Formula I can be prepared in a purity of at least 99%.

In some embodiments, the method of preparing the compound of Formula I includes the method comprising: (a) forming the first reaction mixture comprising the compound of Formula II:

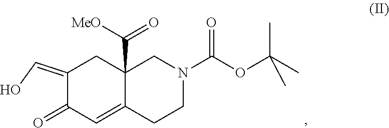 [SMILES: COC(=O)[C@]12C/C(=C/O)C(=O)C=C1CCN(C(=O)OC(C)(C)C)C2]

Mg(OAc)₂·4H₂O in an amount of about 0.6 molar eq. to the compound of Formula II, acetic acid, water, toluene, and 4-fluorophenylhydrazine HCl:

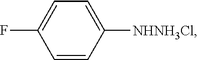

to prepare the compound of Formula I:

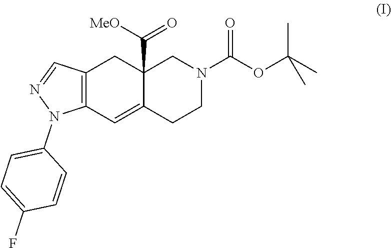 [SMILES: COC(=O)[C@]12Cc3cnn(-c4ccc(F)cc4)c3C=C1CCN(C(=O)OC(C)(C)C)C2]

in a yield of at least 80% and a purity of at least 98%.

### A. Purification of Formula I via Crystallization

The compound of Formula I can be purified by a variety of methods, including crystallization. In some embodiments, the method of preparing the compound of Formula I includes the method further comprising (a1) heating a first crystallization mixture comprising isopropanol, heptane, and the compound of Formula I, such that the compound of Formula I dissolves in the first crystallization mixture; and (a2) cooling the first crystallization mixture to form a first crystalline compound of Formula I.

The first crystalline compound of Formula I can include a variety of impurities, such as, but not limited to, one or more of:

### Impurity A, 6-(tert-butyl) 4a-methyl (4aR,8R)-1-(4-fluorophenyl)-8-hydroxy-1,4,7,8-tetrahydro-6H-pyrazolo[3,4-g]isoquinoline-4a,6(5H)- dicarboxylate

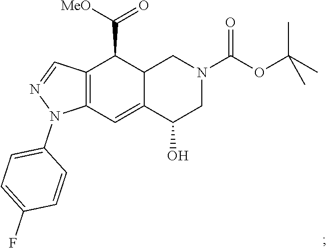 [SMILES: COC(=O)[C@@H]1c2cnn(-c3ccc(F)cc3)c2C=C2C1CN(C(=O)OC(C)(C)C)C[C@@H]2O]

### Impurity C, 6-(tert-butyl) 4a-methyl (R)-2-(4-fluorophenyl)-2,4,7,8-tetrahydro-6H-pyrazolo[3,4-g]isoquinoline-4a,6(5H)-dicarboxylate

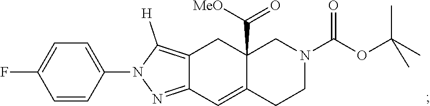 [SMILES: COC(=O)[C@]12Cc3cn(-c4ccc(F)cc4)nc3C=C1CCN(C(=O)OC(C)(C)C)C2]

and

### Impurity D, 6-(tert-butyl) 4a-methyl (R)-1-(4-fluorophenyl)-1,4,7,9-tetrahydro-6H-pyrazolo[3,4-g]isoquinoline-4a,6(5H)-dicarboxylate

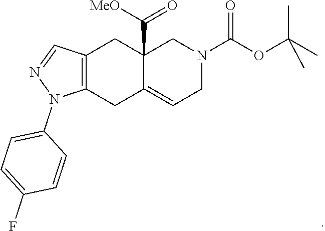 [SMILES: COC(=O)[C@]12Cc3cnn(-c4ccc(F)cc4)c3CC1=CCN(C(=O)OC(C)(C)C)C2]

The impurity present in the compound of Formula I can include Impurity A in an amount of less than 1%. For example, the compound of Formula I can include Impurity A in an amount of less than 1.0%, or less than 0.9, 0.8, 0.75, 0.7, 0.6, 0.5, 0.4, 0.3, 0.25, 0.2, 0.1, 0.09, 0.08, 0.07, 0.06, 0.05, 0.04, 0.03, 0.02, or less than 0.01% of Impurity A. In some embodiments, the compound of Formula I can contain Impurity A in an amount of less than 0.1%. In some embodiments, the compound of Formula I can contain Impurity A in an amount of less than 0.05%.

The impurity present in the compound of Formula I can include Impurity C in an amount of less than 1%. For example, the compound of Formula I can include Impurity C in an amount of less than 1.0%, or less than 0.9, 0.8, 0.75, 0.7, 0.6, 0.5, 0.4, 0.3, 0.25, 0.2, 0.1, 0.09, 0.08, 0.07, 0.06, 0.05, 0.04, 0.03, 0.02, or less than 0.01%. In some embodiments, the compound of Formula I can contain Impurity C in an amount of less than 0.5%. In some embodiments, the compound of Formula I can contain Impurity C in an amount of less than 0.1%. In some embodiments, the compound of Formula I can contain Impurity C in an amount of less than 0.05%.

The impurity present in the compound of Formula I can include Impurity D in an amount of less than 1%. For example, the compound of Formula I can include Impurity D in an amount of less than 1.0%, or less than 0.9, 0.8, 0.75, 0.7, 0.6, 0.5, 0.4, 0.3, 0.25, 0.24, 0.23, 0.22, 0.21, 0.2, or less than 0.1%. In some embodiments, the compound of Formula I can contain Impurity D in an amount of less than 0.25%. In some embodiments, the compound of Formula I can contain Impurity D in an amount of less than 0.2%.

The amount of impurity in the composition containing the compound of Formula I can be measured by any suitable method, such as by weight percentage (w/w), or by HPLC peak area with Ultraviolet detection.

In some embodiments, the method of preparing the compound of Formula I includes the method wherein the compound of Formula I, following step (a2), contains
less than 0.05% of Impurity A:

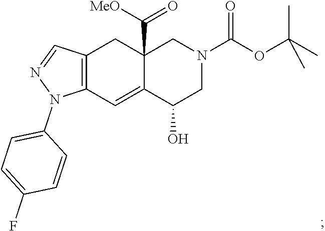 [SMILES: COC(=O)[C@]12Cc3cnn(-c4ccc(F)cc4)c3C=C1[C@@H](O)CN(C(=O)OC(C)(C)C)C2]

less than 0.5% of Impurity C:

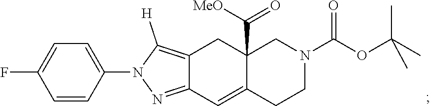 [SMILES: COC(=O)[C@]12Cc3cn(-c4ccc(F)cc4)nc3C=C1CCN(C(=O)OC(C)(C)C)C2]

and
less than 0.25% of Impurity D:

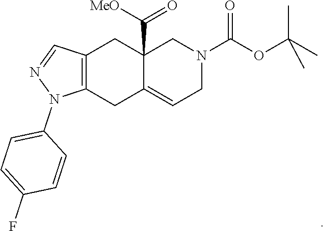 [SMILES: COC(=O)[C@]12Cc3cnn(-c4ccc(F)cc4)c3CC1=CCN(C(=O)OC(C)(C)C)C2]

In some embodiments, the method of preparing the compound of Formula I includes the method further comprising (a3) heating a second crystallization mixture comprising toluene, heptane, and the first crystalline compound of Formula I, such that the first crystalline compound of Formula I dissolves in the second crystallization mixture; and (a4) cooling the second crystallization mixture to form a second crystalline compound of Formula I.

The first and second crystallization mixtures can be heated to any suitable temperature. Representative temperatures include above room temperature, such as, but not limited to, from room temperature to reflux of the first crystallization mixture, from room temperature to 65° C., or from about room temperature to 40° C., or from 40° C. to 65° C., or from 40° C. to 60° C. In some embodiments, the first crystallization mixture can be at a temperature of about 30° C., or at about 35° C., or at about 40° C., or at about 45° C., or at about 50° C., or at about 55° C., or at about 60° C., or at about 65° C.

The first and second crystallization mixture can also be cooled to any suitable temperature. Representative temperatures include below room temperature, at room temperature, or above room temperature.

In some embodiments, the method of preparing the compound of Formula I includes the method wherein the compound of Formula I, following step (a4), contains
less than 0.05% of Impurity A:

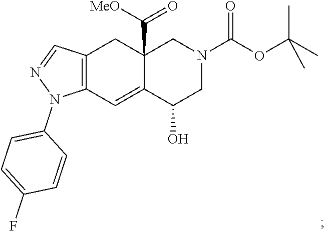 [SMILES: COC(=O)[C@]12Cc3cnn(-c4ccc(F)cc4)c3C=C1[C@@H](O)CN(C(=O)OC(C)(C)C)C2]

less than 0.05% of Impurity C:

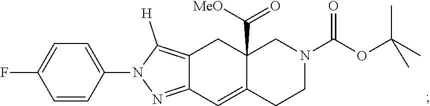 [SMILES: COC(=O)[C@]12Cc3cn(-c4ccc(F)cc4)nc3C=C1CCN(C(=O)OC(C)(C)C)C2]

and
less than 0.20% of Impurity D:

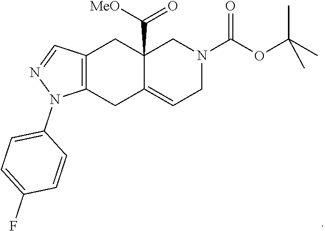 [SMILES: COC(=O)[C@]12Cc3cnn(-c4ccc(F)cc4)c3CC1=CCN(C(=O)OC(C)(C)C)C2]

In some embodiments, the method of preparing the compound of Formula I includes the method comprising:
(a) forming the first reaction mixture comprising the compound of Formula II:

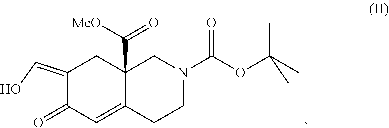 [SMILES: COC(=O)[C@]12C/C(=C/O)C(=O)C=C1CCN(C(=O)OC(C)(C)C)C2]

Mg(OAc)₂·4H₂O in an amount of about 0.6 molar eq. to the compound of Formula II, acetic acid, water, toluene, and 4-fluorophenylhydrazine HCl:

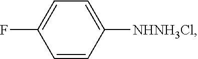

to prepare the compound of Formula I:

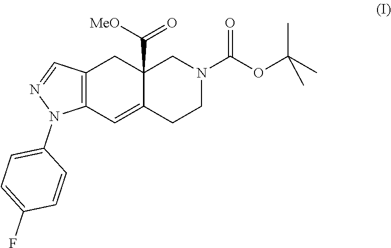 [SMILES: COC(=O)[C@]12Cc3cnn(-c4ccc(F)cc4)c3C=C1CCN(C(=O)OC(C)(C)C)C2]

in a yield of at least 80% and a purity of at least 98%;
(a1) heating the first crystallization mixture comprising isopropanol, heptane, and the compound of Formula I, such that the compound of Formula I dissolves in the first crystallization mixture;
(a2) cooling the first crystallization mixture to form the first crystalline compound of Formula I;
(a3) heating the second crystallization mixture comprising toluene, heptane, and the first crystalline compound of Formula I, such that the first crystalline compound of Formula I dissolves in the second crystallization mixture; and
(a4) cooling the second crystallization mixture to form the second crystalline compound of Formula I,
wherein the second crystalline compound of Formula I contains
less than 0.05% of Impurity A:

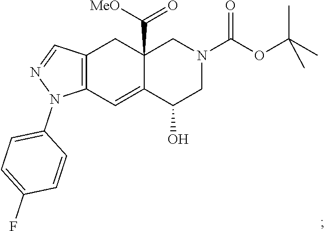 [SMILES: COC(=O)[C@]12Cc3cnn(-c4ccc(F)cc4)c3C=C1[C@@H](O)CN(C(=O)OC(C)(C)C)C2]

less than 0.05% of Impurity C:

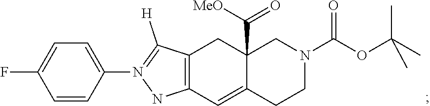

and
less than 0.20% of Impurity D:

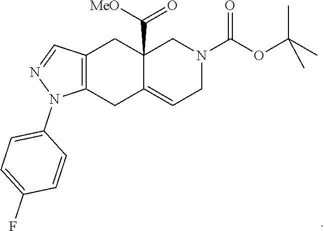 [SMILES: COC(=O)[C@]12Cc3cnn(-c4ccc(F)cc4)c3CC1=CCN(C(=O)OC(C)(C)C)C2]

### B. Preparation of Formula II

The compound of Formula II, 2-(tert-butyl) 8a-methyl (R,Z)-7-(hydroxymethylene)-6-oxo-4,6,7,8-tetrahydroisoquinoline-2,8a(1H,3H)-dicarboxylate:

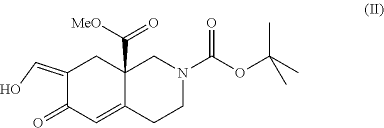 [SMILES: COC(=O)[C@]12C/C(=C/O)C(=O)C=C1CCN(C(=O)OC(C)(C)C)C2]

can be prepared by the method provided in Example 32 of U.S. Pat. No. 7,928,237. In some embodiments, the present invention provides a method of preparing a compound of Formula II:

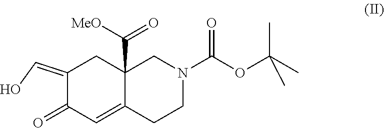 [SMILES: COC(=O)[C@]12C/C(=C/O)C(=O)C=C1CCN(C(=O)OC(C)(C)C)C2]

the method comprising: (c) forming a third reaction mixture comprising an alkylformate, a non-nucleophilic base, and a compound of Formula III:

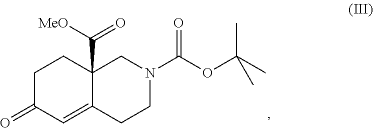 [SMILES: COC(=O)[C@]12CCC(=O)C=C1CCN(C(=O)OC(C)(C)C)C2]

thereby preparing the compound of Formula II.

The alkylformate of the third reaction mixture can have the formula HC(O)O—C₁₋₆ alkyl. Representative alkylformates include, but are not limited to, methylformate, ethylformate, n-propylformate, or n-butylformate.

In some embodiments, the method of preparing the compound of Formula II includes the method wherein the alkylformate is methylformate or ethylformate. In some embodiments, the method of preparing the compound of Formula II includes the method wherein the alkylformate is methylformate.

The alkylformate can be present in any suitable amount to the compound of Formula III. For example, the alkylformate can be present in an amount of from 1 to 10 molar equivalents to the compound of Formula III, or from 1 to 7.5, from 1 to 5, from 2 to 4, from 2.5 to 3.5, from 2.6 to 3.4, from 2.7 to 3.3, from 2.8 to 3.2, or from 2.9 to 3.1 molar equivalents to the compound of Formula III. Representative amounts of the alkylformate include, but are not limited to, about 1 molar equivalents to the compound of Formula III, or about 1.5, 2.0, 2.5, 2.6, 2.7, 2.8, 2.9, 3.0, 3.1, 3.2, 3.3, 3.4, 3.5, 4.0, 4.5, 5.0, 6, 7, 8, 9, or about 10 molar equivalents to the compound of Formula III.

In some embodiments, the method of preparing the compound of Formula II includes the method wherein the methylformate is present in an amount of from 1 to 10 molar eq. to the compound of Formula III. In some embodiments, the method of preparing the compound of Formula II includes the method wherein the methylformate is present in an amount of about 3.0 molar eq. to the compound of Formula III.

The non-nucleophilic base useful in the method of preparing the compound of Formula III includes, but is not limited to, potassium carbonate, sodium carbonate, alkoxides such as potassium tert-butoxide and sodium tert-butoxide, hexamethylsilazane (HMDS), lithium hexamethyldisilazane, sodium hexamethyldisilazine, potassium hexamethyldisilazane, lithium diisopropylamine (LDA), lithium hydride, sodium hydride, potassium hydride, n-butyl lithium, as well as amine bases, such as triethylamine (Et₃N), N,N-diisopropylethylamine (iPr₂NEt; DIPEA), 1,8-diazabicycloundec-7-ene (DBU), 1,5-diazabicyclo(4.3.0)non-5-ene (DBN), 1,4-diazabicyclo[2.2.2]octane (DABCO), N,N-diethylaniline, pyridine, 2,6-lutidine, 2,4,6-collidine, 4-dimethylaminopyridine, and quinuclidine. In some embodiments, the method of preparing the compound of Formula II includes the method wherein the non-nucleophilic base is an alkoxide, hexamethylsilazane (HMDS), lithium hexamethyldisilazane, sodium hexamethyldisilazine, potassium hexamethyldisilazane, lithium diisopropylamine (LDA), lithium hydride, sodium hydride, potassium hydride, or n-butyl lithium. In some embodiments, the method of preparing the compound of Formula II includes the method wherein the non-nucleophilic base is an alkoxide.

The alkoxide useful in the method of preparing the compound of Formula II includes, but is not limited to, methoxide, ethoxide, isopropoxide, tert-butoxide, or tert-pentoxide. Representative counterions for the alkoxides useful in the method of preparing the compound of Formula II includes, but is not limited to, sodium or potassium. In some embodiments, the method of preparing the compound of Formula II includes the method wherein the non-nucleophilic base is sodium tert-butoxide (NaOtBu), sodium tert-pentoxide (NaOtPent), or potassium tert-pentoxide (KOtPent). In some embodiments, the method of preparing the compound of Formula II includes the method wherein the non-nucleophilic base is sodium tert-pentoxide (NaOtPent).

The alkoxide can be present in any suitable amount to the compound of Formula III. For example, the alkoxide can be present in an amount of from 1 to 10 molar equivalents to the compound of Formula III, or from 1 to 7.5, from 1 to 5, from 2 to 4, from 2.5 to 3.5, from 2.6 to 3.4, from 2.7 to 3.3, from 2.8 to 3.2, or from 2.9 to 3.1 molar equivalents to the compound of Formula III. Representative amounts of the alkylformate include, but are not limited to, about 1 molar equivalents to the compound of Formula III, or about 1.5, 2.0, 2.5, 2.6, 2.7, 2.8, 2.9, 3.0, 3.1, 3.2, 3.3, 3.4, 3.5, 4.0, 4.5, 5.0, 6, 7, 8, 9, or about 10 molar equivalents to the compound of Formula III. In some embodiments, the method of preparing the compound of Formula II includes the method wherein the sodium tert-pentoxide is present in an amount of about 3.0 molar equivalents to the compound of Formula III.

The third reaction mixture can also include an inorganic salt. Representative inorganic salts include, but are not limited to, lithium chloride, lithium bromide, lithium iodide, sodium chloride, sodium bromide, sodium iodide, potassium chloride, potassium bromide, or potassium iodide. In some embodiments, the method of preparing the compound of Formula II includes the method wherein the third reaction mixture includes lithium chloride.

The inorganic salt can be present in any suitable amount to the compound of Formula III. For example, the inorganic salt can be present in an amount of from 0.1 to 10 molar equivalents to the compound of Formula III, or from 0.1 to 5, from 0.2 to 4, from 0.5 to 3.5, from 0.5 to 3.0, from 0.5 to 2.0, or from 1.0 to 1.4 molar equivalents to the compound of Formula III. Representative amounts of the inorganic salt include, but are not limited to, about 1 molar equivalents to the compound of Formula III, or about 1.0, 1.1, 1.2, 1.3, 1.4, 1.5, 1.6, 1.7, 1, 1.8, 1.9, 2, 3, 4, 5, 6, 7, 8, 9 or about 10 molar equivalents to the compound of Formula III. In some embodiments, the method of preparing the compound of Formula II includes the method wherein the lithium chloride is present in an amount of about 1.2 molar equivalents to the compound of Formula III.

In some embodiments, the method of preparing the compound of Formula II includes the method wherein the third reaction mixture further includes a third solvent. Any suitable solvent can be used as the third solvent in the third reaction mixture. The third solvent can include, but is not limited to, pentanes, hexanes, heptane, benzene, toluene, diethyl ether, tetrahydrofuran, 2-methyltetrahydrofuran, acetone, ethyl acetate, acetonitrile, methylene chloride, and chloroform. In some embodiments, the method of preparing the compound of Formula II includes the method wherein the third solvent includes diethyl ether, tetrahydrofuran, or 2-methyltetrahydrofuran. In some embodiments, the method of preparing the compound of Formula II includes the method wherein the third reaction mixture further includes 2-methyltetrahydrofuran.

Conditions suitable to prepare the compound of Formula I can include any suitable time and temperature, as described below in the examples section.

The third reaction mixture can be cooled to any suitable temperature. Representative temperatures include at or below room temperature. Representative temperatures include below room temperature, such as, but not limited to, from −78° C. to room temperature, from −78° C. to 0° C., from −60° C. to −10° C., from −50° C. to −20° C., or from −40° C. to −30° C. Other temperatures of the third reaction mixture include, but are not limited to, about −78° C., or about −70° C., −60° C., −50° C., −40° C., −30° C., −20° C., −10° C., or about 0° C. In some embodiments, the method of preparing the compound of Formula II includes the method wherein the third reaction mixture is at a temperature of about −40° C.

The compound of Formula II can include a variety of impurities, such as, but not limited to, Impurity X, 2-(tert-butyl) 8a-methyl (R,4E,7Z)-4,7-bis(hydroxymethylene)-6-oxo-4,6,7,8-tetrahydroisoquinoline-2,8a(1H,3H)-dicarboxylate:

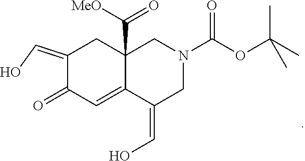 [SMILES: COC(=O)[C@]12C/C(=C/O)C(=O)C=C1/C(=C\O)CN(C(=O)OC(C)(C)C)C2]

The impurity present in the compound of the compound of Formula II can include Impurity X in an amount of less than 1%. For example, the compound of Formula II can include Impurity X in an amount of less than 1.0%, or less than 0.9, 0.8, 0.75, 0.7, 0.6, 0.5, 0.4, 0.3, 0.25, 0.2, 0.1, 0.09, 0.08, 0.07, 0.06, 0.05, 0.04, 0.03, 0.02, or less than 0.01%. In some embodiments, the compound of Formula II can contain Impurity X in an amount of less than 0.1%. In some embodiments, the compound of Formula II can contain Impurity X in an amount of less than 0.01%.

In some embodiments, the method of preparing the compound of Formula II includes the method wherein the compound of Formula II contains Impurity X in an amount of less than 0.01%:

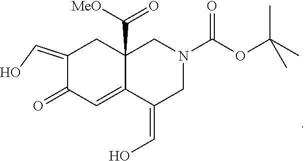 [SMILES: COC(=O)[C@]12C/C(=C/O)C(=O)C=C1/C(=C\O)CN(C(=O)OC(C)(C)C)C2]

In some embodiments, the method of preparing the compound of Formula II includes the method including (c) forming the third reaction mixture comprising methylformate in an amount of about 3.0 molar eq. to the compound of Formula III, sodium tert-pentoxide, lithium chloride, 2-methyltetrahydrofuran, and the compound of Formula III:

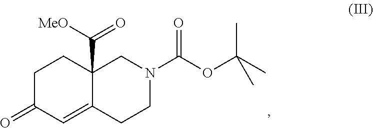 [SMILES: COC(=O)[C@]12CCC(=O)C=C1CCN(C(=O)OC(C)(C)C)C2]

wherein the third reaction mixture is at a temperature of about −40° C.,
thereby preparing the compound of Formula II:

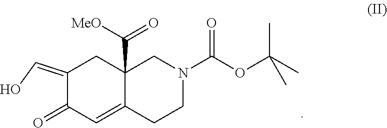 [SMILES: COC(=O)[C@]12C/C(=C/O)C(=O)C=C1CCN(C(=O)OC(C)(C)C)C2]

In some embodiments, the method of preparing the compound of Formula I includes the method wherein the compound of Formula II is prepared by the methods of the present invention.

In some embodiments, the method of preparing the compound of Formula II includes the method including (c) forming the third reaction mixture comprising methylformate in an amount of about 3.0 molar eq. to the compound of Formula III, sodium tert-pentoxide, lithium chloride, 2-methyltetrahydrofuran, and the compound of Formula III:

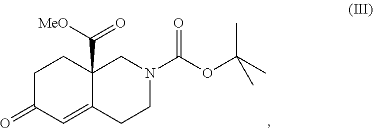 [SMILES: COC(=O)[C@]12CCC(=O)C=C1CCN(C(=O)OC(C)(C)C)C2]

wherein the third reaction mixture is at a temperature of about −40° C.,
thereby preparing the compound of Formula II:

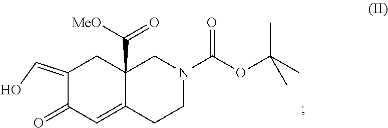 [SMILES: COC(=O)[C@]12C/C(=C/O)C(=O)C=C1CCN(C(=O)OC(C)(C)C)C2]

and
(a) forming the first reaction mixture comprising the compound of Formula II, Mg(OAc)₂·4H₂O in an amount of about 0.6 molar eq. to the compound of Formula II, acetic acid, water, toluene, and 4-fluorophenylhydrazine HCl:

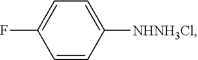

to prepare the compound of Formula I:

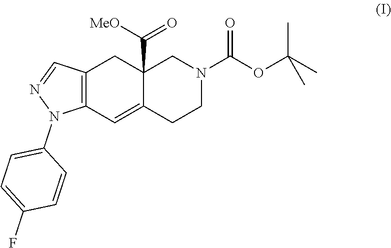 [SMILES: COC(=O)[C@]12Cc3cnn(-c4ccc(F)cc4)c3C=C1CCN(C(=O)OC(C)(C)C)C2]

in a yield of at least 80% and a purity of at least 98%.

In some embodiments, the method of preparing the compound of Formula II includes the method also including:
(a1) heating the first crystallization mixture comprising isopropanol, heptane, and the compound of Formula I, such that the compound of Formula I dissolves in the first crystallization mixture;
(a2) cooling the first crystallization mixture to form the first crystalline compound of Formula I;
(a3) heating the third crystallization mixture comprising toluene, heptane, and the first crystalline compound of Formula I, such that the first crystalline compound of Formula I dissolves in the third crystallization mixture; and
(a4) cooling the third crystallization mixture to form the third crystalline compound of Formula I,
wherein the third crystalline compound of Formula I contains
less than 0.05% of Impurity A:

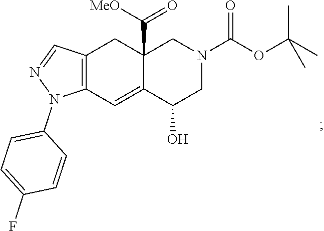 [SMILES: COC(=O)[C@]12Cc3cnn(-c4ccc(F)cc4)c3C=C1[C@@H](O)CN(C(=O)OC(C)(C)C)C2]

less than 0.05% of Impurity C:

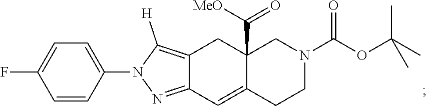 [SMILES: COC(=O)[C@]12Cc3cn(-c4ccc(F)cc4)nc3C=C1CCN(C(=O)OC(C)(C)C)C2]

and
less than 0.20% of Impurity D:

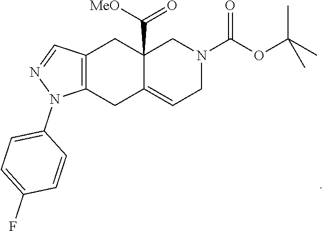 [SMILES: COC(=O)[C@]12Cc3cnn(-c4ccc(F)cc4)c3CC1=CCN(C(=O)OC(C)(C)C)C2]

### C. Preparation of Formula III

The compound of Formula III, 2-(tert-butyl) 8a-methyl (R)-6-oxo-4,6,7,8-tetrahydroisoquinoline-2,8a(1H,3H)-dicarboxylate:

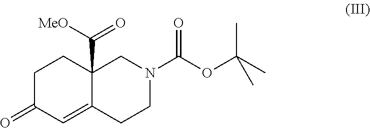 [SMILES: COC(=O)[C@]12CCC(=O)C=C1CCN(C(=O)OC(C)(C)C)C2]

can be prepared by the method in Example 31 of U.S. Pat. No. 7,928,237.

In some embodiments, the method of preparing the compound of Formula II includes the method wherein the compound of Formula III is prepared by the method comprising: (d) forming a fourth reaction mixture comprising pyrrolidine, acetic acid, and the compound of Formula IV:

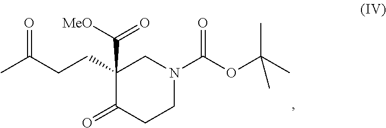 [SMILES: COC(=O)[C@@]1(CCC(C)=O)CN(C(=O)OC(C)(C)C)CCC1=O]

thereby preparing the compound of Formula III:

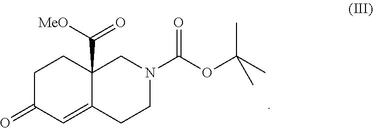 [SMILES: COC(=O)[C@]12CCC(=O)C=C1CCN(C(=O)OC(C)(C)C)C2]

The pyrrolidine can be present in any suitable amount to the compound of Formula IV. For example, the pyrrolidine can be present in an amount of from 0.05 to 1.0 molar equivalents to the compound of Formula IV, or from 0.1 to 1.0, or from 0.1 to 0.5 molar equivalents to the compound of Formula IV. Representative amounts of the pyrrolidine include, but are not limited to, about 0.1 molar equivalents to the compound of Formula IV, or about 0.2, 0.3, 0.4, 0.5, 0.6, 0.7, 0.8, 0.9, or about 1 molar equivalents to the compound of Formula IV. In some embodiments, the method of preparing the compound of Formula III includes the method wherein the pyrrolidine is present in an amount of about 0.3 molar equivalents to the compound of Formula IV.

The acetic acid can be any suitable acetic acid. For example, the acetic acid can be glacial acetic acid. The acetic acid can be present in any suitable amount to the compound of Formula IV. For example, the acetic acid can be present in an amount of from 0.1 to 10 molar equivalents to the compound of Formula IV, or from 0.5 to 5, or from 1 to 2, or from 1 to 1.5 molar equivalents to the compound of Formula IV. Representative amounts of the acetic acid include, but are not limited to, about 0.5 molar equivalents to the compound of Formula IV, or about 0.6, 0.7, 0.8, 0.9, 1.0, 1.1, 1.2, 1.3, 1.4, or about 1.5 molar equivalents to the compound of Formula IV. In some embodiments, the method of preparing the compound of Formula III includes the method wherein the acetic acid is present in an amount of about 1.5 molar equivalents to the compound of Formula IV.

In some embodiments, the method of preparing the compound of Formula III includes the method wherein the fourth reaction mixture further includes a fourth solvent. Any suitable solvent can be used as the fourth solvent in the fourth reaction mixture. The fourth solvent can include, but is not limited to, methanol, ethanol, n-propanol, isopropanol, diethyl ether, tetrahydrofuran, 2-methyltetrahydrofuran, and methyl t-butyl ether. The fourth solvent can include, but is not limited to, methanol, ethanol, n-propanol, isopropanol, diethyl ether, tetrahydrofuran, 2-methyltetrahydrofuran, methyl t-butyl ether, and toluene. In some embodiments, the method of preparing the compound of Formula III includes the method wherein the fourth solvent includes isopropanol, methyl t-butyl ether, and combinations thereof. In some embodiments, the method of preparing the compound of Formula III includes the method wherein the fourth reaction mixture further includes methyl t-butyl ether.

The compound of Formula III can be purified by a variety of methods, including crystallization. In some embodiments, the method of preparing the compound of Formula III includes the method further comprising forming a fourth crystallization mixture comprising isopropanol and the compound of Formula III, such that the compound of Formula III dissolves in the fourth crystallization mixture; and adding water to the fourth crystallization mixture to form the crystalline compound of Formula III.

The ratio of isopropanol to water can be any suitable ratio. For example, the ratio of isopropanol to water can be from 1:1 to 1:10 (vol/vol), or from 1:1 to 2:10, or from 1:2 to 3:10 (vol/vol). Representative ratios of the isopropanol to water includes, but is not limited to, about 1:8 (vol/vol), or about 1:7, 1:6, 1:5, 1:4, 3:8, 4:8, 5:8, or about 6:8 (vol/vol). In some embodiments, the method of preparing the compound of Formula III includes the fourth crystallization mixture having isopropanol and water in a 3:8 (vol/vol).

In some embodiments, the method of preparing the compound of Formula II includes the method wherein the compound of Formula III is prepared by the method comprising: (d) forming a fourth reaction mixture comprising pyrrolidine in an amount of 0.3 molar equivalents to the compound of Formula IV, glacial acetic acid in an amount of 1.2 molar equivalents to the compound of Formula IV, isopropanol, and the compound of Formula IV:

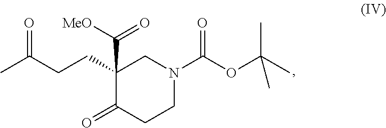 [SMILES: COC(=O)[C@@]1(CCC(C)=O)CN(C(=O)OC(C)(C)C)CCC1=O]

thereby preparing the compound of Formula III:

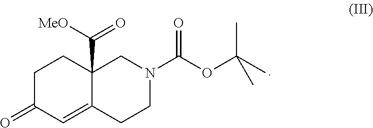 [SMILES: COC(=O)[C@]12CCC(=O)C=C1CCN(C(=O)OC(C)(C)C)C2]

### D. Preparation of Formula IV

The compound of Formula IV, 1-(tert-butyl) 3-methyl (S)-4-oxo-3-(3-oxobutyl)piperidine-1,3-dicarboxylate:

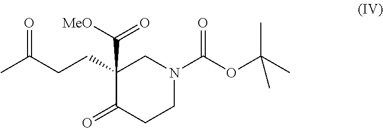 [SMILES: COC(=O)[C@@]1(CCC(C)=O)CN(C(=O)OC(C)(C)C)CCC1=O]

can be prepared by the method in Example 30 of U.S. Pat. No. 7,928,237.

In some embodiments, the method of preparing the compound of Formula II includes the method wherein the compound of Formula IV is prepared by the method comprising: (e) forming a fifth reaction mixture comprising a compound of Formula V:

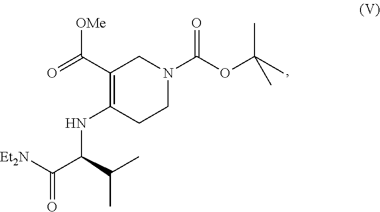

methyl vinyl ketone (MVK) in an amount of about 1.6 molar eq. to the compound of Formula V, Cu(OAc)2 (anhydrous), and dimethylformamide, at a temperature of from 10 to 25° C., thereby preparing the compound of Formula IV:

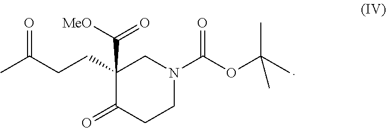 [SMILES: COC(=O)[C@@]1(CCC(C)=O)CN(C(=O)OC(C)(C)C)CCC1=O]

The fifth reaction mixture can be at any suitable temperature. Representative temperatures include, but are not limited to, from 0° C. to 50° C., from 5° C. to 40° C., from 10° C. to 30° C., from 15° C. to 25° C., from 18° C. to 25° C., or from 20° C. to 23° C. Other temperatures of the third reaction mixture include, but are not limited to, about 0° C., or about 10° C., 15° C., 16° C., 17° C., 18° C., 19° C., 20° C., 21° C., 22° C., 23° C., 24° C., 25° C., 26° C., 27° C., 28° C., 29° C., or about 30° C. In some embodiments, the method of preparing the compound of Formula IV includes the method wherein the fifth reaction mixture has a temperature of from 20 to 23° C.

In some embodiments, the method of preparing the compound of Formula IV includes the method further comprising: (e1) adding to the fifth reaction mixture an aqueous mixture comprising HCl and LiCl, under conditions suitable to prepare the compound of Formula IV.

In some embodiments, the method of preparing the compound of Formula IV includes the method wherein the compound of Formula IV contains less than 5% of MVK polymer. In some embodiments, the method of preparing the compound of Formula IV includes the method wherein the compound of Formula IV contains less than 1% of MVK polymer. In some embodiments, the method of preparing the compound of Formula IV includes the method wherein the compound of Formula IV contains less than 0.1% of MVK polymer. In some embodiments, the method of preparing the compound of Formula IV includes the method wherein the compound of Formula IV contains less than 0.05% of MVK polymer. The amound of MVK polymer present with the compound of Formula IV can be determined by weight percentage (w/w) or by HPLC peak area with ultraviolet detection.

### IV. LOW IMPURITY COMPOSITIONS

The present invention provides compositions of Formula I having a low impurity content. The impurity content can be expressed in a variety of different methods. For example, the impurity content can be expressed as a weight percentage (w/w), or by % (HPLC peak area). In some embodiments, the impurity content can be expressed as a % (HPLC peak area). In some embodiments, the present invention provides a composition comprising: a compound of Formula I in an amount of at least 99%:

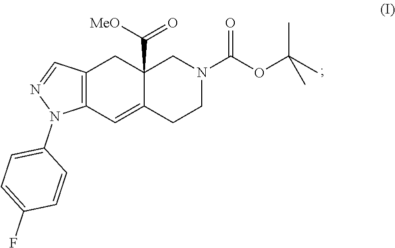 [SMILES: COC(=O)[C@]12Cc3cnn(-c4ccc(F)cc4)c3C=C1CCN(C(=O)OC(C)(C)C)C2]

and
one or more impurity in an amount of from 0.01 to 1%.

The composition of Formula I can include one or more impurities present in a total amount of from 0.01 to 1%.

The impurity present in the composition of the compound Formula I can include Impurity A in an amount of less than 1%. For example, the composition of the compound Formula I can include less than 1.0%, or less than 0.9, 0.8, 0.75, 0.7, 0.6, 0.5, 0.4, 0.3, 0.25, 0.2, 0.1, 0.09, 0.08, 0.07, 0.06, 0.05, 0.04, 0.03, 0.02, or less than 0.01% of Impurity A. In some embodiments, the composition comprising the compound of Formula I can contain less than 0.1% of Impurity A. In some embodiments, the composition comprising the compound of Formula I can contain less than 0.05% of Impurity A.

The impurity present in the composition of the compound Formula I can include Impurity C in an amount of less than 1%. For example, the composition of the compound Formula I can include less than 1.0%, or less than 0.9, 0.8, 0.75, 0.7, 0.6, 0.5, 0.4, 0.3, 0.25, 0.2, 0.1, 0.09, 0.08, 0.07, 0.06, 0.05, 0.04, 0.03, 0.02, or less than 0.01% of Impurity C. In some embodiments, the composition comprising the compound of Formula I can contain less than 0.5% of Impurity C. In some embodiments, the composition comprising the compound of Formula I can contain less than 0.1% of Impurity C. In some embodiments, the composition comprising the compound of Formula I can contain less than 0.05% of Impurity C.

The impurity present in the composition of the compound Formula I can include Impurity D in an amount of less than 1%. For example, the composition of the compound Formula I can include less than 1.0%, or less than 0.9, 0.8, 0.75, 0.7, 0.6, 0.5, 0.4, 0.3, 0.25, 0.24, 0.23, 0.22, 0.21, 0.2, or less than 0.1% of Impurity D. In some embodiments, the composition comprising the compound of Formula I can contain less than 0.25% of Impurity D. In some embodiments, the composition comprising the compound of Formula I can contain less than 0.2% of Impurity D.

In some embodiments, the compound of Formula I includes the compound wherein the impurity includes at least one of:
less than 0.05% of Impurity A:

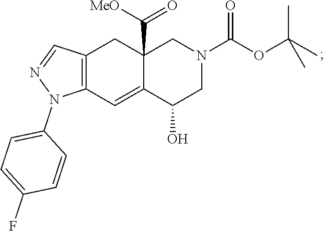 [SMILES: COC(=O)[C@]12Cc3cnn(-c4ccc(F)cc4)c3C=C1[C@@H](O)CN(C(=O)OC(C)(C)C)C2]

less than 0.05% of Impurity C:

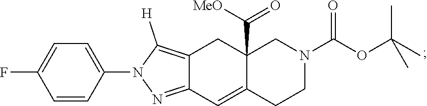 [SMILES: COC(=O)[C@]12Cc3cn(-c4ccc(F)cc4)nc3C=C1CCN(C(=O)OC(C)(C)C)C2]

and
less than 0.20% of Impurity D:

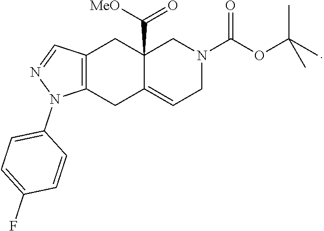 [SMILES: COC(=O)[C@]12Cc3cnn(-c4ccc(F)cc4)c3CC1=CCN(C(=O)OC(C)(C)C)C2]

In some embodiments, the compound of Formula I includes the compound prepared by the methods of the present invention.

### V. EXAMPLES

The following abbreviations are used in the methods below:

| | |
|---|---|
| ° C. | Degree Celsius |
| ACN, MeCN | acetonitrile |
| AcOH | Acetic acid |
| AcONa | Sodium acetate |
| aq | Aqueous |
| atm | Atmospheric pressure |
| Barg | Bar(g); Pressure in bar as determined by gauge |
| Boc | t-butyloxycarbonyl (t-BuOC(O)—) |
| DIPEA | N,N-diisopropylethylamine (iPr₂NEt) |
| DMAP | 4-dimethylaminopyridine |
| EtOAc | Ethyl Acetate |
| eq or equiv | Equivalent |
| FA | Formic acid (HC(O)OH) |
| g | Gram |
| HDPE | High density polyethylene |
| IPA | Isopropyl alcohol (2-propanol) |
| Kg | kilogram |
| L | Liter |
| M | Molar |
| mbarg | Millibar gauge pressure |
| mins | minutes |
| mL | milliliter |
| MSA | Methanesulfonic acid |
| MTBE | methyl t-butyl ether |
| NLT | Not Less Than |
| nm | nanometers |
| NMR | Nuclear Magnetic Resonance |
| PMI | Potential Mutagenic Impurity |
| ppm | Parts per million |
| r.t. | room temperature |
| SEC | Size Exclusion Chromatography |
| TEA | triethylamine (Et₃N) |
| THF | tetrahydrofuran |
| TMEDA | N,N,N′,N-Tetramethylethylenediamine |
| μm | micrometer |

X-ray Powder Diffraction (XRPD). XRPD analyses were performed using a Panalytical Xpert Pro diffractometer equipped with a Cu X-ray tube and a Pixcel detector system. The isothermal samples were analysed in transmission mode and held between low density polyethylene films. The XRPD program used included the following parameters: (1) range 3-40° 2θ, (2) step size 0.013°, (3) counting time 99 sec, and (4) about 22 min run time. XRPD patterns were sorted using HighScore Plus 2.2c software.

Differential Scanning Calorimetry (DSC). DSC analyses were carried out on a Perkin Elmer Jade Differential Scanning Calorimeter. Accurately weighed samples were placed in crimped aluminium pans. Each sample was heated under nitrogen at a rate of 10° C./minute to a maximum of 300° C. Indium metal was used as the calibration standard. Temperatures were reported at the transition onset to the nearest 0.01 degree.

The reaction steps of the present invention can be performed for any suitable reaction time. For example, the reaction time can be for minutes, hours, or days. In some embodiments, the reaction time can be for several hours, such as at least eight hours. In some embodiments, the reaction time can be for several hours, such as at least overnight. In some embodiments, the reaction time can be for several days. In some embodiments, the reaction time can be for at least two hours. In some embodiments, the reaction time can be for at least eight hours. In some embodiments, the reaction time can be for at least several days. In some embodiments, the reaction time can be for about two hours, or for about 4 hours, or for about 6 hours, or for about 8 hours, or for about 10 hours, or for about 12 hours, or for about 14 hours, or for about 16 hours, or for about 18 hours, or for about 20 hours, or for about 22 hours, or for about 24 hours. In some embodiments, the reaction time can be for about 1 day, or for about two days, or for about three days, or for about four days, or for about five days, or for about six days, or for about a week, or for about more than a week.

The reaction steps of the present invention can be performed at any suitable reaction temperature. Representative temperatures include, but are not limited to, below room temperature, at room temperature, or above room temperature. Other temperatures useful in the methods of the present invention include from about −40° C. to about 65° C., or from about room temperature to about 40° C., or from about 40° C. to about 65° C., or from about 40° C. to about 60° C. In some embodiments, the reaction mixture can be at a temperature of about room temperature, or at a temperature of about 15° C., or at about 20° C., or at about 25° C. or at about 30° C., or at about 35° C., or at about 40° C., or at about 45° C., or at about 50° C., or at about 55° C., or at about 60° C., or at about 65° C.

### Example 1. Preparation of Formula V (Compound 6), 1-(tert-butyl) 3-methyl (S)-4-((1-(diethylamino)-3-methyl-1-oxobutan-2-yl)amino)-5,6-dihydropyridine-1,3(2H)-dicarboxylate

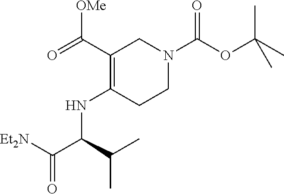

To a 100 L vessel, previously rinsed with MTBE was added (tert-butoxycarbonyl)-L-valine (Compound 1) [3.5 kg, 16.11 mol, 1.0 eq.] and MTBE [25.9 kg, 35 L, 10 vols.] with stirring at 25° C. After 88 min, the solid had dissolved. The vessel was then cooled to 0° C., and Pivaloyl chloride [2.14 kg, 17.74 mol, 1.1 eq.] followed by N-methylmorpholine [2.45 kg, 24.21 mol, 1.5 eq.] were charged to the reactor at −5˜5° C. After the addition was complete, the reactor was heated to 20° C. The resulting mixture was aged for 3 hours at 20° C., where the conversion of Compound 1 to the pivolate ester (mixed anhydride) was determined by HPLC.

The reaction mixture was then cooled to 0° C. and diethylamine [2.14 kg, 17.74 mol, 1.1 eq.] was charged to the reactor over a period of 20 minutes, maintaining the temperature at −5-5° C. The resulting mixture was aged for 10 minutes at 20° C. Warmed to 20° C. The resulting mixture was aged for 5 hours at 20° C., where the area of Compound 1 was analyzed by HPLC. 1 N Hydrochloric acid solution [21.00 kg, 17.5 L, 5 vols.] was charged to the vessel, stirred for 15 minutes, and aged for 15 minutes, the biphasic mixture was allowed to settle, and the aqueous layer drained. Kept the organic layer in the reactor. 5 wt % Sodium Hydrogen Carbonate [11.03 kg, 10.5 L, 3 vols.] was charged to the vessel, stirred for 15 minutes, and aged for 15 minutes, the biphasic mixture was allowed to settle, and the aqueous layer drained from the reactor, keeping the organic phase in the reactor.

Purified water [7 kg, 7 L, 2 vols.] was then charged to the vessel containing the organic phase and the biphasic mixture stirred for 15 min. Stirrig was stopped and the mixture was aged for 15 min to allow the biphasic mixture to settle. The aqueous layer was separated and the organic phase was concentrated to approximately 14.0-17.5 L [˜4.5 vols.] under reduced pressure, maintaining a batch temperature below 40° C. Isopropyl alcohol [13.83 kg, 17.5 L, 5 vols.] was charged to the vessel and concentrated to approximately 14 L to 17.5 L [4.5 vols.] under reduced pressure, maintaining a batch temperature below 55° C. Isopropyl alcohol [13.83 kg, 17.5 L, 5 vols.] was charged to the vessel and concentrated to approximately 14.0-17.5 L [˜4.5 vols.] under reduced pressure, maintaining a batch temperature below 55° C. HPLC analysis of the resulting isopropy acetate solution contained 3.95 kg (90% assay yield) of tert-butyl (S)-(1-(diethylamino)-3-methyl-1-oxobutan-2-yl)carbamate (Compound 2) with a chemical purity of 97.3 area %

A 50 L vessel was dried under vacuum prior to use. The IPA solution of Compound 2 [tert-butyl (S)-(1-(diethylamino)-3-methyl-1-oxobutan-2-yl)carbamate; 3.66 kg, 13.44 moles, 1.11 eq.] was charged to the vessel over 20 min at 20-21° C. IPA [7.90 kg, 10.0 L, 3 vols.] was charged to the vessel to make up the total volume to 16.5 L [5 vols.] over 20 min at 5-10° C. 95 wt % conc. H₂SO₄ [1.40 kg, 14.24 moles, 1.11 eq.] was added over 50 min at 7-17° C. (H₂SO₄ was alternatively added over about 10 hours or longer.) Warmed to 60° C. The resulting mixture was aged for 29 hours at 60-61° C.

Cool to 0° C. over 45 min. Purified water [5.12 kg, 5.12 L, 1.55 vols.] was charged to the vessel over 15 minutes at 0° C. TMEDA [3.40 kg, 29.24 mol, 2.28 eq.] was charged to the vessel over 40 minutes at 0-4° C. 1-(tert-butyl) 3-methyl 4-oxopiperidine-1,3-dicarboxylate (Compound 5) [3.3 kg, 12.83mol, 1.0 eq.] was charged to the vessel over 50 minutes at −5-3° C. The batch was warmed to 60° C. over 50 minutes and aged for 8 hours at 60° C.

The batch was cooled to 25° C. and seeded with Compound 6 (9.9 g, 0.038 moles, 0.3 wt %) and then aged for 1 hour. Purified water [15.84 kg, 15.84 L, 4.8 vols.] was added over 33 minutes at 25° C. and the batch was aged for 30 minutes. The batch was cooled to 0° C. and aged for 1 hour at 0° C. The batch was filtered and deliquored under nitrogen pressure. The mixture of IPA [2.6 kg, 3.3 L, 1 vol.] and purified water [6.6 kg, 6.6 L, 2 vols.] was charged to the vessel and subsequently transferred into the oyster filter as the first cake wash. Purified water [6.6 kg, 6.6 L, 2 vols.] was charged as the second cake wash. The cake was dried under vacuum at 40° C. for 20 hours with a minimal nitrogen sweep and analyzed for weight percent. 4.0 kg of Compound 6-was obtained.

### Example 2. Preparation of Formula IV (Compound 7), 1-(tert-butyl) 3-methyl (S)-4-oxo-3-(3-oxobutyl)piperidine-1,3-dicarboxylate

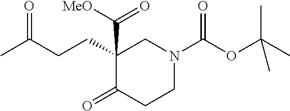 [SMILES: COC(=O)[C@@]1(CCC(C)=O)CN(C(=O)OC(C)(C)C)CCC1=O]

A 400 L vessel was dried under vacuum prior to use. Compound 6 [11 kg] and Cu(OAc)₂ [0.97 kg, 20 mol %] were charged to the vessel and degassed three times under vacuum. DMF [3 vols] was charged to the vessel, degassed three times under positive pressure of nitrogen, and stirred at 35° C. for 10 minutes. The batch was cooled to 21° C. and MVK, 90% [3.33 kg, 1.6 eq] was charged over 97 minutes maintaining batch temperature 22° C. After addition of MVK, the vessel was subjected to 2 +500 mBArg pressure purges. The batch was aged for 22 hours under stirring and conversion of Compound 6 analysed by HPLC. HPLC showed 96.9 A % conversion of Compound 6.

The batch was cooled to 1° C. and methyl tert-butyl ether [81.4 kg, 110 L, 10 vol] charged. Maintaining temperature below 11° C., ˜0.5 M HCl/10 wt % LiCl [116.6 kg, 10 vol] was charged to the vessel. After addition, the vessel was subjected to 2 +500 mBArg pressure purges. The batch was warmed to 22° C. and aged for 22 minutes under rapid stirring before being sampled and analysed for the hydrolysis of the imine intermediate to form Compound 7 by HPLC. HPLC showed 97.3A % conversion (2.7 LCAP imine intermediate remained) to Compound 7.

The biphasic mixture was allowed to settle and the aqueous layer drained. The organic layer was drained to a second drum for temporary storage. The aqueous layer and methyl tert-butyl ether [40.7 kg, 55 L, 5 vol] were charged to the vessel, stirred and aged for 5 minutes. After addition and during stirring, the vessel was subjected to 2 +500 mBArg pressure purges. The biphasic mixture was allowed to settle and the aqueous layer drained. The first organic cut was charged into the vessel with the second organic cut. After addition and during stirring, the vessel was subjected to 2 +500 mBArg pressure purges.

10 wt % Lithium Chloride [23.2 kg, 22 L, 2 vol] was charged to the vessel. After addition and during stirring, the vessel was subjected to 2 +500 mBArg pressure purges. The wash was aged for 3 minutes, the biphasic mixture was allowed to settle, and the aqueous layer drained. 5 wt % Sodium Hydrogen Carbonate [23.2 kg, 22 L, 2 vol] was charged to the vessel. After addition and during stirring, the vessel was subjected to 2 +500 mBArg pressure purges. The wash was aged for 2 minutes, the biphasic mixture was allowed to settle, and the aqueous layer drained. The batch was cooled to 0° C. and Diethylamine [4.11 kg, 2.1 eq] was charged over 13 minutes maintaining temperature below 5° C. The batch was warmed to 22° C. and aged for 31 minutes then sampled and analysed for scavenging of excess MVK. HPLC showed 0.6 LCAP MVK remaining.

The batch was cooled to 0° C. and 1 M Hydrochloric Acid solution [67.3 kg, 6 vol] was charged maintaining temperature below 10° C. The batch was warmed to 22° C., aged for 2 minutes, the biphasic mixture was allowed to settle, and the aqueous layer drained. 10 wt % Lithium Chloride [23.2 kg, 22 L, 2 vol] was charged to the vessel, stirred, aged for 2 minutes, the biphasic mixture was allowed to settle, and the aqueous layer drained. 3 wt % Sodium Hydrogen Carbonate [23.2 kg, 22 L, 2 vol] was charged to the vessel, stirred, aged for 7 minutes, the biphasic mixture was allowed to settle, and the aqueous layer drained. The organic layer was drained. Solution contained 8.43 kg Compound 7 (96% assay yield, 94.6 LCAP at 210nm, 99.8% cc) in 114 kg methyl tert-butyl ether solution (6.89 wt %). Solution was stored at 2-8° C.

### Example 3. Preparation of Formula III (Compound 8), 2-(tert-butyl) 8a-methyl (R)-6-oxo-4,6,7,8-tetrahydroisoquinoline-2,8a(1H,3H)-dicarboxylate

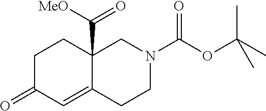 [SMILES: COC(=O)[C@]12CCC(=O)C=C1CCN(C(=O)OC(C)(C)C)C2]

A 400 L vessel was dried under vacuum prior to use. Charged solution of Compound 7 [8.25 kg, 120.1 kg in MTBE] to the reactor. Concentrated to 3 volumes [˜26 L] maintaining internal temperature below 40° C. Charged IPA [19.6 kg, 3 vols.]. Concentrated to 3 volumes [˜25 L] maintaining internal temperature below 40° C. Charged IPA [52.3 kg, 8 vols]. Cooled the contents of the reactor to 1° C. Charged Acetic acid glacial [1.84 kg, 1.2 eq] maintaining an internal temperature below 10° C. Charged Pyrrolidine [0.543 kg, 0.3 eq] maintaining an internal temperature below 10° C. 563 grams was charged which corresponds to a 3.8% overage. The contents of the reactor were warmed to 20° C. and aged at 20-21° C. for 18.2 h, then analysed for reaction conversion.

Aqueous NH₄Cl [10% wt/wt; 69.2 kg, 8 vols.] was charged to the reactor and the reaction mixture was aged at 25° C. for at least 18 hours. The mixture was then concentrated to 7 volumes [˜61 L], maintaining an internal temperature below 40° C. Next, methyl tert-butylether [61.6 kg, 10 vols] was charged to the vessel and the mixture stirred for 5 minutes. Stirring was stopped and the biphasic mixture was allowed to settle. The phases were separated and the organic layer was washed with water [16.6 kg, 2 vols]. The organic phase was then washed twice with a 10% wt/wt solution of K₃PO₄ in water [45.8 kg; 5 vols]. The organic phase was then washed with water [16.6 kg, 2 vols]. HPLC analysis of the isolated organic phase showed that the MTBE solution contained 7.40 kg of compound of Formula III, which corresponded to a 95% assay yield. Subsequent processing operations, solvent charges and volumes involving this solution were based upon this 95% assay yield.

The MTBE solution containing the Compound of Formula III was concentrated to 3 volumes with respect to Assay yield [˜22 L], maintaining internal temperature below 40° C. IPA [52.2 kg, 9 vols] was charged to the reactor, and the resulting solution was again concentrated to 3 volumes [˜22 L]. Water [14.5 kg, 2 volumes] was then charged to the reactor over a period of 30 minutes, followed by seeding of the mixture with a sample of the Compound of Formula III (39.0 g; ˜0.5% wt/wt relative to the assay value, 7.4 kg) at 22° C. and age for 1 h18 min. A second portion of water [7.24 kg, 1 vol] was then charged to the reactor over a period of 32 minutes and the resulting seedbed aged at 22° C. for at least 16.25 h. A third portion of water [36.2 kg, 5 vol] was charged over 1.3 h and the resulting seedbed aged for 1 h. The reactor contents were then cooled 0° C. over a period of 1.3 h then aged for an additional 1 h. The slurry was then filtered and the filter cake was washed with 3 volumes of a 1:4 mixture (v/v) of IPA [4.34 kg; 0.5 vols] and water [17.4 kg; 2 vols]. The resulting solid was then dried in the oven at 30° C. under a considerable flow of nitrogen, until the majority of the water had been removed. The solid was further dried in the oven at 40° C., under a nitrogen sweep, to give 6.79 kg of Formula III, which corresponded to an 87% isolate yield. HPLC analysis of the dry solid showed the material to have a assay value of 99.9% wt/wt and a chemical purity of 99.7 area %. Karl Fischer (KF) titration showed the solid to contain 0.08% wt/wt of water.

### Example 4. Preparation of Formula II (Compound 8a), 2-(tert-butyl) 8a-methyl (R,Z)-7-(hydroxymethylene)-6-0x0-4,6,7,8-tetrahydroisoquinoline-2,8a(1H,3H)- dicarboxylate

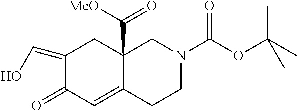 [SMILES: COC(=O)[C@]12C/C(=C/O)C(=O)C=C1CCN(C(=O)OC(C)(C)C)C2]

Charged Compound 8 (50 g, 1.0 equiv.) and lithium chloride (1.2 equiv.) to a 2 L three-neck round bottom flask at ambient temperature, with an overhead stirrer and degas by three times vacuum/N₂ cycles. Degassed 2-MeTHF (10 vol.) added, then degas the solution sparging with N₂ for 5 min. Methyl formate (4.0 equiv.) was added. The contents of the flask were cooled to −40° C. while being agitated with overhead stirring. NaOtPent (2.5M in THF) (3.0 equiv.) was added over 30 min maintaining an internal temperature below −40° C. The reaction mixture was aged at −40° C. for 1.5 hr. The reaction was sampled and analysed via HPLC using the X-Bridge analytical method, showing 2A % Compound 8 remaining.

The reaction mixture was quenched with a solution of AcOH (3.5 equiv.) in 2-MeTHF (5 vol.), maintaining an internal temperature below-35° C. The Reaction mixture was warmed to 5° C. then diluted with H₂O (10 vol.). The organic was washed with then H₂O (5 vol.). The organic stream was assayed and analysed via HPLC using the X-Bridge and FFP2 analytical methods:
X-Bridge: Compound 8a 88.7%, 96.2 LCAP, Compound 8 2.0%, 2.1 LCAP, dimer 0.9 LCAP.
FFP2: no Impurity X detected.

Compound 8a was extracted into the aqueous with 10wt % K₃PO₄ (15 vol.), then (3 vol.). Toluene (15 vol.) was added to the K₃PO₄ layer, followed by 6M HCl (2.5 vol.) portion-wise, keeping an eye on the internal temperature and pH. Aqueous layer acidified to pH 5. The organic acidic toluene stream was washed with H₂O (2×5 vol.). The organic stream was assayed and analysed via HPLC using the X-Bridge and FFP2 analytical methods:
X-Bridge: Compound 8a 86.2%, 99.4 LCAP, Compound 8 <0.1%
FFP2: no Impurity X detected.

### Example 5. Preparation of Formula I (Compound 9), 6-(tert-butyl) 4a-methyl (R)-1-(4-fluorophenyl)-1,4,7,8-tetrahydro-6H-pyrazolo[3,4-glisoquinoline-4a,6(5H)-dicarboxylate

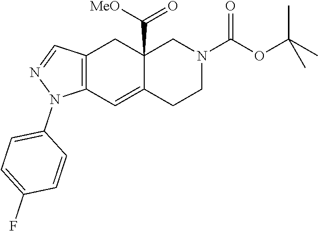 [SMILES: COC(=O)[C@]12Cc3cnn(-c4ccc(F)cc4)c3C=C1CCN(C(=O)OC(C)(C)C)C2]

A 180 L vessel was dried under vacuum prior to use. Charged solution of Compound 8a (6.5 kg; 1.0 equiv.) in Toluene (ca. 6.00 wt %) to the reactor. Concentrated to 10 volumes [˜60 L] maintaining a batch temperature of ≤40° C. Charged acetic acid (10.4 kg; 9.0 equiv.) followed by water (0.3 vols.). Degassed the contents of the reactor via vacuum nitrogen cycle. Charged magnesium acetate tetrahydrate (2.58 kg; 0.6 equiv.) and stirred the contents of the reactor at 20° C. for 15 minutes. Charged 4-fluorophenylhydrazine⋅HCl (3.45 kg; 1.1 equiv.) maintaining internal temperature <25° C. Aged the reaction at 20° C. for 18 hours and 31 minutes and analysed for conversion. HPLC analysis indicated 100% conversion (0.0 A % Compound 8a with respect to Compound 9).

Charged water (29.7 kg, 5 vols.), aged for 1 hour and 10 minutes at 20-22° C. and separated the layers formed. Washed the organic phase with 1 M aq. HCl (30.3 kg, 5 vols.) twice. Washed the organic phase with 10 wt % aq K₃PO₄ (31.2 kg, 5 vols.). Washed the organic phase 5 wt % aq NaCl (30.8 kg, 5 vols.). Wash the organic phase with water (30.1 kg, 5 vols.).

Discharged the organic phase and determined the assay yield and LCAP of Compound 9. HPLC analysis of the organic stream indicated there were 7.47 kg of Compound 9 corresponding to an assay yield of 99.6%, in 98.7 LCAP (0.03 LCAP Impurity A, 1.07 LCAP Impurity C, 0.24 LCAP Impurity D). MVK Polymer analysis indicated 1.84 A % of SEC impurities.

### Example 6. First Crystallization of Formula I (Compound 9)

Rinsed the reactor with IPA (30.0 kg) and discharged as waste. Charged the organic stream to the reactor via an in-line filter and concentrates to 3 volumes with respect to Compound 9 [˜22 L] under reduced pressure, keeping T<40° C. Charged IPA (60.0 kg, 10 vols. w.r.t Compound 9) to the reactor. Concentrated to 3 volumes w.r.t Compound 9 [˜23 L] under reduced pressure, keeping T<40° C. Charge IPA (6.4 kg, 1 vol. w.r.t Compound 9) to the reactor. Heated the resulting solution to 58.6° C., aged until homogeneous and analysed for toluene content by NMR. Cooled the stirred solution to 50° C. and charged seeds (41 g Compound 9; 0.55 wt %). Cooled to 45° C. and aged the seedbed for 30 minutes at 46° C. Charged Heptane (20.5 kg, 4 vols. w.r.t Compound 9) over 1 hour and 42 minutes at 45° C. Cooled the resulting slurry to 21° C. over 2 hours 8 minutes. Aged the slurry at 20° C. for 14 hours 53 minutes. Cooled the slurry to 0° C. over 14 minutes using upper and lower vessel jackets at −15° C. and aged at 0° C. for 1 hour 21 minutes. Filtered and washed with 3 volumes of 0° C. 1:1 volume mixture of IPA (8.8 kg, 1.5 vols.) and Heptane (7.7 kg, 1.5 vols.). Dewatered the cake with nitrogen for 30 minutes.

Dried in the oven at 40° C. with a nitrogen sweep for 20 hours 36 minutes. 6.66 kg (88% yield, 99.2 wt %) of Compound 9 was obtained in 99.4 A % LCAP (0.02 LCAP Impurity A; 0.41 LCAP Impurity C; 0.19 LCAP Impurity D). Impurity C levels in the dry cake were not homogenous. An 80 g sample was dissolved and analysed and found to contain 0.03 A % Impurity A, 0.50 LCAP Impurity C, 0.20 Impurity D. MVK Polymer analysis indicated 0.13 A %.

### Example 7. Second Crystallization of Formula I (Compound 9)

A 180 L vessel was dried under vacuum prior to use. Charged Compound 9 (6.52 kg) to the reactor. Charged toluene (14.1 kg, 2.5 vols. w.r.t Compound 9) and heptane (11.4 kg, 2.5 vols. w.r.t Compound 9). Warmed the contents of the reactor to 69° C. and aged until homogenous. Cooled the stirred solution to 54° C., charged seeds (32 g, 0.5 wt %) and aged for 1 hour 40 minutes. Charged heptane (22.0 kg, 5 vols. w.r.t. Compound 9) over 2 hours 37 minutes. Cooled the contents of the reactor to 20° C. over 4 hours 16 minutes and aged for a subsequent 10 hours 51 minutes. Filtered and washed with 3 volumes of 20° C. 1:3 volume mixture of toluene (3.5 kg) and heptane (8.4 kg). Dried in the oven at 40° C. with a nitrogen sweep for 20 hours 36 minutes. 5.65 kg (75% yield) of Compound 9 was obtained in 99.9 A % LCAP (0.03 LCAP Impurity A; 0.02 LCAP Impurity C; 0.14 LCAP Impurity D). An additional 2% of Compound 9 material formed a dome on the top of the bottom outlet value. MVK Polymer analysis indicated <0.05 Impurity A %.

TABLE 1
Impurity Data for Product of Example 4 to Example 7

| | | | Formula I | Formula I |
|---|---|---|---|---|
| | | | (Compound 9) | (Compound 9) |
| | Formula II | Formula I | First | Second |
| | (Compound 8a) | (Compound 9) | Crystallization | Crystallization |
| Impurity | Example 4 | Example 5 | Example 6 | Example 7 |
| A | — | 0.03% | 0.03% | 0.03% |
| C | — | 1.07% | 0.50% | 0.02% |
| D | — | 0.24% | 0.20% | 0.14% |
| X | ND | — | — | — |
| MVK Polymer | NA | 1.84% | 0.13% | <0.05% |
| Percent Yield | 86.2% | 99.6% | 88.5% | 86.7% |
| Purity (HPLC) | 99.4% | 98.7% | 99.4% | 99.9% |

*Analytical method: HPLC with Ultraviolet detection. ND: Not Detected. NA: Not Available.

### Example 8. Alternative Preparation of Formula I (Compound 9), 6-(tert-butyl) 4a-methyl (R)-1-(4-fluorophenyl)-1.4.7.8-tetrahydro-6H-pyrazolo[3.4-glisoquinoline-4a,6(5H)-dicarboxylate

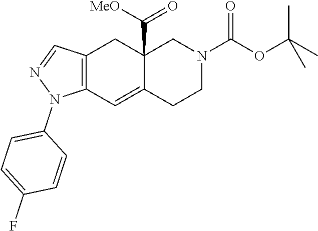 [SMILES: COC(=O)[C@]12Cc3cnn(-c4ccc(F)cc4)c3C=C1CCN(C(=O)OC(C)(C)C)C2]

Compound 9 can also be prepared according to the methods of Examples 30 to 33 in U.S. Pat. No. 7,928,237.

Formula II (Compound 8a), 2-(tert-butyl) 8a-methyl (R,Z)-7-(hydroxymethylene)-6-oxo-4,6,7,8-tetrahydroisoquinoline-2,8a(1H,3H)-dicarboxylate

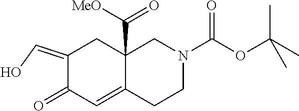 [SMILES: COC(=O)[C@]12C/C(=C/O)C(=O)C=C1CCN(C(=O)OC(C)(C)C)C2]

Methyl tert-butyl ether (232.0 kg) was charged to the 3000 L stainless steel reactor at 15-25° C. and sampled for KF analysis to ensure it was ≤0.1%. Hexamethyldisilazane (102.0 kg) was added into the reactor at 15-25° C. The stirrer was started. Oxygen content was detected to confirm it was ≤0.1%. The mixture was cooled to −40 to −20° C. under the protection with nitrogen. Oxygen content was detected to confirm it was ≤0.1%.

Maintaining the temperature at −40 to −20° C., n-butyllithium (2.5 mol/L, 174.2 kg) was added to the reactor. N,N,N,′N-Tetramethylethylenediamine (73.4 kg) was added drop wise to the mixture at a rate of 50-80 kg/h at −40 to −20° C. After addition, the mixture was maintained at −40 to −20° C. for 30 min.

The methyl tert-butyl ether solution of Compound 8 (628.0 kg solution, 65.0 kg corrected) was added to the mixture at −40 to −20° C. After addition, the mixture was stirred for 1-2 h. The mixture was cooled to −60 to −50° C.

Methyl tert-butyl ether (156.0 kg) was added to the 3000 L glass-lined reactor at 15-25° C. and sampled for KF analysis to ensure it was ≤0.1%. 2,2,2-Trifluoroethyl formate (60.6 kg) was added to the 3000 L glass-lined reactor at 15-25° C. The solution in the 300 L glass-lined reactor was transferred to the 3000 L stainless steel reactor at −60 to −40° C. under nitrogen protection.

The mixture was reacted at −60 to −40° C. After 1 h, the mixture was sampled every 1-2 h for HPLC analysis to ensure area % of Compound 8 was ≤15% or the difference between two consecutive samples was ≤1%.

A part of solution (810.0 kg) of hydrochloric acid in purified water was added to the mixture at T≤0° C. The addition was stopped when the temperature rose to over 0° C. The aqueous phase was sampled for pH to ensure it was 6-7 (FIO).

Hydrochloric acid (250.0 kg) in water (1254.0 kg) Solution (580.0 kg) of hydrochloric acid in purified water was added to the mixture at 0-20° C. The aqueous phase was sampled for pH to ensure it was 3-5 (FIO). The mixture was stirred for 0.5-1 h and settled for 0.5-1 h at 15-25° C. before separation. A solution (682.6 kg) of potassium carbonate in purified water was added to the organic phase at 0-10° C. to adjust pH to 9-10.

The mixture was stirred for 0.5-1 h and settled for 0.5-1 h at 15-25° C. before separation. Methyl tert-butyl ether (190.0 kg) was added to the aqueous phase for extraction.

The mixture was stirred for 0.5-1h and settled for 0.5-1 h at 15-25° C. before separation. All the organic phase was combined.

The combined organic phase was washed by potassium carbonate solution of Ins. 15 (225.0 kg) at 15-25° C. The mixture was stirred for 0.5-1 h and settled for 0.5-1 h before separation. A solution (850.0 kg) of hydrochloric acid in purified water was added to the aqueous phase at 0-10° C. The aqueous phase was sampled for pH to ensure it was 3-4.

The mixture was extracted by dichloromethane (232.2 kg×2) twice at 15-25° C. In each extraction, the mixture was stirred for 0.5-1 h and settled for 0.5-1 h before separation. A solution of sodium chloride (95.6 kg) in water (464.0 kg) was added to the combined organic phase. The mixture was stirred for 0.5-1 h and settled for 0.5-1 h at 15-25° C. before separation. The organic phase was sampled for purity and Compound8a wt % analysis.

### Formula I (Compound 9), 6-(tert-butyl) 4a-methyl (R)-1-(4-fluorophenyl)-1,4,7,8-tetrahydro-6H-pyrazolo[3,4-g]isoquinoline-4a,6(5H)-dicarboxylate

Compound 8a DCM solution (1133.4 kg, 119.3 kg corrected) was charged to the 3000 L glass-lined reactor 1. Then the mixture was concentrated under reduced pressure (P≤-0.06 MPa) until 130-195 L mixture left. Acetic acid (597.8 kg) was added to the mixture at 20-40° C. and stirred until the mixture was clear. The mixture was adjusted to 15-25° C., sodium acetate (37.8 kg) was added to the mixture at 15-25° C. and the mixture was stirred for 1-1.5 h.

4-Fluorophenylhydrazine hydrochloride (84.0 kg, 75.6 kg corrected) was added to the mixture at 15-25° C. in several portions with 4-6 kg for every portion and 20-40 min interval. The mixture was reacted at 15-25° C. After 1 h, the mixture was sampled every 1-4 h for HPLC analysis to ensure area % of Compound 8a was ≤1 area %.

The mixture was transferred into a 5000 L glass-lined reactor 2. Purified water (1309.0 kg) and methyl tert-butyl ether (1206.0 kg) were added to the reactor 1 at 15-25° C. Then the mixture was stirred for 0.5 h and then transferred into the reactor 2.

The mixture was settled until layered at 15-25° C. before separation. The aqueous phase was extracted by methyl tert-butyl ether (869.4 kg). The mixture was stirred for NLT 20-40 min and settled until layered at 15-25° C. before separation.

A solution of potassium carbonate was added to wash the combined organic phase at 15-25° C. The mixture was stirred for 20-40 min and settled until layered at 15-25° C. before separation. While maintaining the temperature at 15-25° C. The organic phase was washed with potassium carbonate solution. The aqueous phase was sampled for pH to ensure it was >7. The mixture was stirred and settled until layered at 15-25° C. before separation.

The organic phase was filtered through stainless steel nutsche filter which was pre-loaded with silica gel (31.2 kg). The filter cake was rinsed with methyl tert-butyl ether (339.8 kg+339.1 kg+337.4 kg) three times, the nutsche filter was soaked 0.5-1 h every time. The filtrate was concentrated at T≤40° C. under reduced pressure (P≤-0.06 MPa) until 455-520 L left.

The mixture was adjusted to 35-45° C., then n-heptane (265.2 kg) was added to the concentrated mixture at 35-45° C. at the reference rate of 20-45 kg/h. After addition, the mixture was stirred for 2-3 h. The mixture was cooled to 15-25° C. at the reference rate of 3-5° C./h.

The mixture was stirred at 15-25° C. for crystallization. After 2-3 h, the mixture was sampled every 2-3 h for mother liquor wt % analysis until it was ≤4.0%, or the difference between two consecutive samples was ≤0.5%. The mixture was filtered with a Φ1250 stainless steel centrifuge. The filter cake was rinsed with n-heptane (176.8 kg).

TABLE 2
Impurity Data for Product of Example 8

| | | | Formula I | Formula I | Formula I |
|---|---|---|---|---|---|
| | Formula II | | (Compound 9) | (Compound 9) | (Compound 9) |
| Impurity | (Compound 8a)^{‡} | | Crude^{‡} | Pure [Lot A] | Pure [Lot B] |
| A | — | | 1.04% (0.76-1.44%) | 0.70% | 0.67% |
| C | — | | 0.32% (0.23-0.47%) | 0.06% | 0.02% |
| D | — | | ND | ND | 0.01% |
| X | NA | | — | — | — |
| MVK Polymer | NA | | 2.47% (0.49-6.06%) | ND | ND |
| Percent Yield | 59% | (57-61%) | 81.5% (76.6-84.8%) | 58.2% | 73.5% |
| Purity (HPLC) | 91.8% | (87.3-95.7%) | 98.1% (97.3-98.5%) | 99.2% | 99.3% |

* Analytical method: HPLC with Ultraviolet detection. ND: Not Detected. NA: Not Available.
^{†}Average values reported for the purity, impurity profile, and percent yield for the three batches of crude Compound 9 obtained from the reaction of Compound 8a with 4-fluorophenylhydrazine hydrochloride. Values in parentheses correspond to the range of results obtained for the three batches of crude Compound 9.
^{‡}Average values for yield and purity reported for the eight batches of Compound 8a manufactured from Compound 8. Values in parentheses correspond to the range of results for yield and purity obtained for each of the eight Compound 8a batches.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of preparing a compound of Formula I: 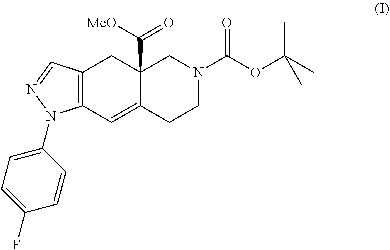 [SMILES: COC(=O)[C@]12Cc3cnn(-c4ccc(F)cc4)c3C=C1CCN(C(=O)OC(C)(C)C)C2]
comprising:
(a) forming a first reaction mixture comprising a compound of Formula II: 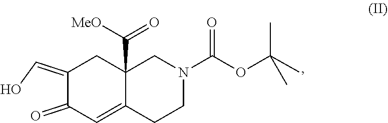 [SMILES: COC(=O)[C@]12C/C(=C/O)C(=O)C=C1CCN(C(=O)OC(C)(C)C)C2]
Mg(OAc)₂, and 4-fluorophenylhydrazine HCl: 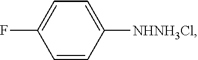
under conditions suitable to prepare the compound of Formula I in a yield of at least 80% and a purity of at least 98%.

2. (canceled)

3. The method of claim 1, wherein the Mg(OAc)₂ is present in an amount of from 0.5 to 1.0 molar eq. to the compound of Formula II.

4. (canceled)

5. (canceled)

6. The method of claim 1, further comprising
(a1) heating a first crystallization mixture comprising isopropanol, heptane, and the compound of Formula I, such that the compound of Formula I dissolves in the first crystallization mixture; and
(a2) cooling the first crystallization mixture to form a first crystalline compound of Formula I.

7. The method of claim 6, wherein the compound of Formula I, following step (a2), contains
less than 0.05% of Impurity A: 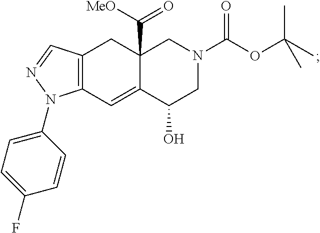 [SMILES: COC(=O)[C@]12Cc3cnn(-c4ccc(F)cc4)c3C=C1[C@@H](O)CN(C(=O)OC(C)(C)C)C2]
less than 0.5% of Impurity C: 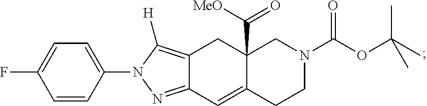 [SMILES: COC(=O)[C@]12Cc3cn(-c4ccc(F)cc4)nc3C=C1CCN(C(=O)OC(C)(C)C)C2]
and
bless than 0.25% of Impurity D:

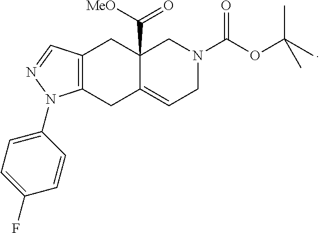 [SMILES: COC(=O)[C@]12Cc3cnn(-c4ccc(F)cc4)c3CC1=CCN(C(=O)OC(C)(C)C)C2]

8. The method of claim 6, further comprising
(a3) heating a second crystallization mixture comprising toluene, heptane, and the first crystalline compound of Formula I, such that the first crystalline compound of Formula I dissolves in the second crystallization mixture; and
(a4) cooling the second crystallization mixture to form a second crystalline compound of Formula I.

9. (canceled)

10. The method of claim 1, comprising:
(a) forming the first reaction mixture comprising the compound of Formula II: 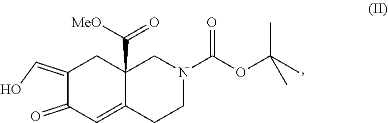 [SMILES: COC(=O)[C@]12C/C(=C/O)C(=O)C=C1CCN(C(=O)OC(C)(C)C)C2]
Mg(OAc)₂·4H₂O in an amount of about 0.6 molar eq. to the compound of Formula II, acetic acid, water, toluene, and 4-fluorophenylhydrazine HCl: 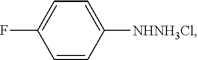
to prepare the compound of Formula I: 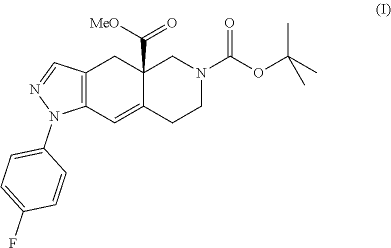 [SMILES: COC(=O)[C@]12Cc3cnn(-c4ccc(F)cc4)c3C=C1CCN(C(=O)OC(C)(C)C)C2]
in a yield of at least 80% and a purity of at least 98%;
(a1) heating the first crystallization mixture comprising isopropanol, heptane, and the compound of Formula I, such that the compound of Formula I dissolves in the first crystallization mixture;
(a2) cooling the first crystallization mixture to form the first crystalline compound of Formula I;
(a3) heating the second crystallization mixture comprising toluene, heptane, and the first crystalline compound of Formula I, such that the first crystalline compound of Formula I dissolves in the second crystallization mixture; and
(a4) cooling the second crystallization mixture to form the second crystalline compound of Formula I,
wherein the second crystalline compound of Formula I contains
less than 0.05% of Impurity A: 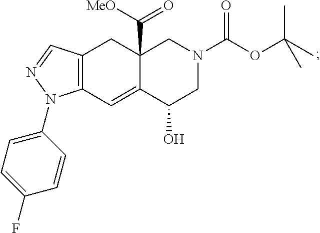 [SMILES: COC(=O)[C@]12Cc3cnn(-c4ccc(F)cc4)c3C=C1[C@@H](O)CN(C(=O)OC(C)(C)C)C2]
less than 0.05% of Impurity C: 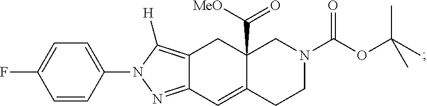 [SMILES: COC(=O)[C@]12Cc3cn(-c4ccc(F)cc4)nc3C=C1CCN(C(=O)OC(C)(C)C)C2]
and
less than 0.20% of Impurity D:

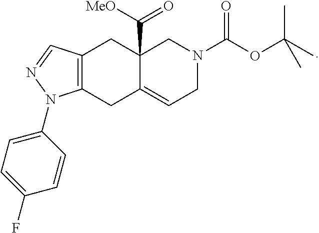 [SMILES: COC(=O)[C@]12Cc3cnn(-c4ccc(F)cc4)c3CC1=CCN(C(=O)OC(C)(C)C)C2]

11. A method of preparing a compound of Formula II: 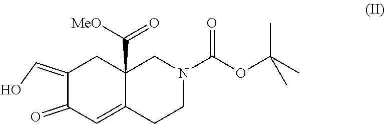 [SMILES: COC(=O)[C@]12C/C(=C/O)C(=O)C=C1CCN(C(=O)OC(C)(C)C)C2]
the method comprising:
(c) forming a third reaction mixture comprising an alkylformate, a non-nucleophilic base, and a compound of Formula III: 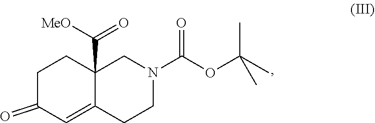 [SMILES: COC(=O)[C@]12CCC(=O)C=C1CCN(C(=O)OC(C)(C)C)C2]
thereby preparing the compound of Formula II.

12. The method of claim 11, wherein the alkylformate is methylformate or ethylformate.

13. (canceled)

14. The method of claim 11, wherein the alkylformate is methylformate and is present in an amount of from 1 to 10 molar eq. to the compound of Formula III.

15. (canceled)

16. The method of claim 11, wherein the non-nucleophilic base is an alkoxide, hexamethylsilazane (HMDS), lithium hexamethyldisilazane, sodium hexamethyldisilazine, potassium hexamethyldisilazane, lithium diisopropylamine (LDA), lithium hydride, sodium hydride, potassium hydride, or n-butyl lithium.

17. The method of claim 11, wherein the non-nucleophilic base is sodium tert-butoxide (NaOtBu), sodium tert-pentoxide (NaOtPent), or potassium tert-pentoxide (KOtPent).

18. (canceled)

19. (canceled)

20. The method of claim 11, wherein
(c) forming the third reaction mixture comprising methylformate in an amount of about 3.0 molar eq. to the compound of Formula III, sodium tert-pentoxide, lithium chloride, 2-methyltetrahydrofuran, and the compound of Formula III: 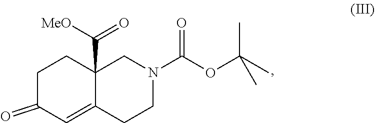 [SMILES: COC(=O)[C@]12CCC(=O)C=C1CCN(C(=O)OC(C)(C)C)C2]
wherein the third reaction mixture is at a temperature of about −40° C.,
thereby preparing the compound of Formula II:

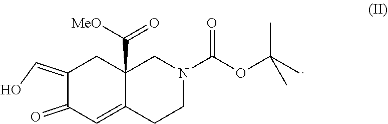 [SMILES: COC(=O)[C@]12C/C(=C/O)C(=O)C=C1CCN(C(=O)OC(C)(C)C)C2]

21. The method of claim 1, wherein the compound of Formula II is prepared by the method of claim 11.

22. The method of claim 21, comprising:
(c) forming the third reaction mixture comprising methylformate in an amount of about 3.0 molar eq. to the compound of Formula III, sodium tert-pentoxide, lithium chloride, 2-methyltetrahydrofuran, and the compound of Formula III: 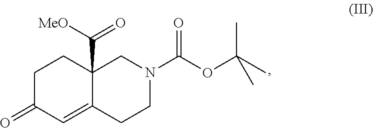 [SMILES: COC(=O)[C@]12CCC(=O)C=C1CCN(C(=O)OC(C)(C)C)C2]
wherein the third reaction mixture is at a temperature of about −40° C.,
thereby preparing the compound of Formula II: 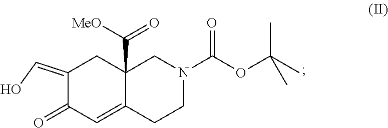 [SMILES: COC(=O)[C@]12C/C(=C/O)C(=O)C=C1CCN(C(=O)OC(C)(C)C)C2]
and
(a) forming the first reaction mixture comprising the compound of Formula II,
Mg(OAc)₂·4H₂O in an amount of about 0.6 molar eq. to the compound of Formula II, acetic acid, water, toluene, and 4-fluorophenylhydrazine HCl: 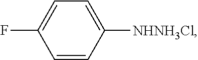
to prepare the compound of Formula I: 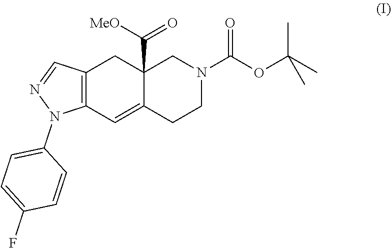 [SMILES: COC(=O)[C@]12Cc3cnn(-c4ccc(F)cc4)c3C=C1CCN(C(=O)OC(C)(C)C)C2]
in a yield of at least 80% and a purity of at least 98%.

23. The method of 22, further comprising:
(a1) heating the first crystallization mixture comprising isopropanol, heptane, and the compound of Formula I, such that the compound of Formula I dissolves in the first crystallization mixture;
(a2) cooling the first crystallization mixture to form the first crystalline compound of Formula I;
(a3) heating the third crystallization mixture comprising toluene, heptane, and the first crystalline compound of Formula I, such that the first crystalline compound of Formula I dissolves in the third crystallization mixture; and
(a4) cooling the third crystallization mixture to form the third crystalline compound of Formula I,
wherein the third crystalline compound of Formula I contains
less than 0.05% of Impurity A: 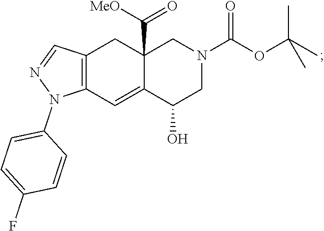 [SMILES: COC(=O)[C@]12Cc3cnn(-c4ccc(F)cc4)c3C=C1[C@@H](O)CN(C(=O)OC(C)(C)C)C2]
less than 0.05% of Impurity C: 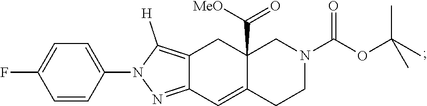 [SMILES: COC(=O)[C@]12Cc3cn(-c4ccc(F)cc4)nc3C=C1CCN(C(=O)OC(C)(C)C)C2]
and
less than 0.20% of Impurity D:

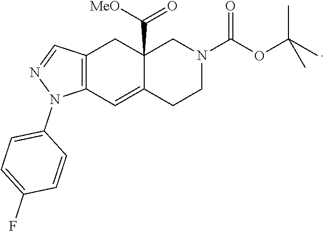 [SMILES: COC(=O)[C@]12Cc3cnn(-c4ccc(F)cc4)c3C=C1CCN(C(=O)OC(C)(C)C)C2]

24. The method of claim 11, wherein the compound of Formula III is prepared by the method comprising:
(d) forming a fourth reaction mixture comprising pyrrolidine, acetic acid, and the compound of Formula IV: 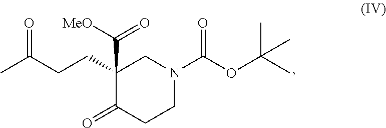 [SMILES: COC(=O)[C@@]1(CCC(C)=O)CN(C(=O)OC(C)(C)C)CCC1=O]
thereby preparing the compound of Formula III:

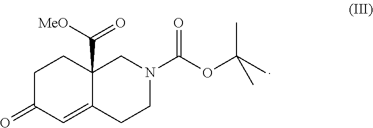 [SMILES: COC(=O)[C@]12CCC(=O)C=C1CCN(C(=O)OC(C)(C)C)C2]

25. The method of claim 24, wherein the compound of Formula IV is prepared by the method comprising:
(e) forming a fifth reaction mixture comprising a compound of Formula V: 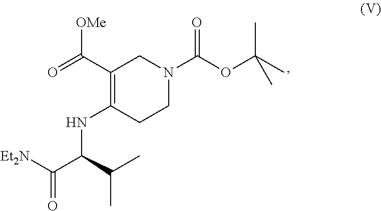
methyl vinyl ketone (MVK) in an amount of about 1.6 molar eq. to the compound of Formula V, Cu(OAc)2 (anhydrous), and dimethylformamide, at a temperature of from 10 to 25° C., thereby preparing the compound of Formula IV:

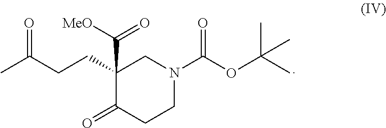 [SMILES: COC(=O)[C@@]1(CCC(C)=O)CN(C(=O)OC(C)(C)C)CCC1=O]

26. The method of claim 25, wherein the fifth reaction mixture has a temperature of from 20 to 23° C.

27. The method of claim 25, further comprising:
(e1) adding to the fifth reaction mixture an aqueous mixture comprising HCl and LiCl, under conditions suitable to prepare the compound of Formula IV.

28. The method of claim 25, wherein the compound of Formula IV contains less than 0.1% (w/w) of MVK polymer.

29. (canceled)

30. A composition comprising:
a compound of Formula I in an amount of at least 99%: 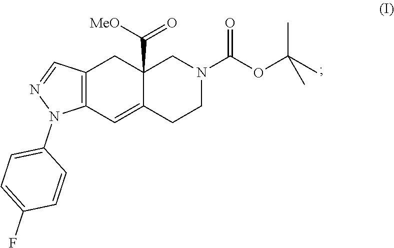 [SMILES: COC(=O)[C@]12Cc3cnn(-c4ccc(F)cc4)c3C=C1CCN(C(=O)OC(C)(C)C)C2]
and
one or more impurity in an amount of from 0.01 to 1%.

31. The composition of claim 30, wherein the composition comprises:
less than 0.05% of Impurity A: 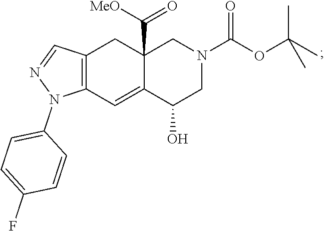 [SMILES: COC(=O)[C@]12Cc3cnn(-c4ccc(F)cc4)c3C=C1[C@@H](O)CN(C(=O)OC(C)(C)C)C2]
less than 0.05% of Impurity C: 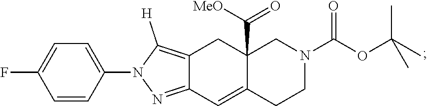 [SMILES: COC(=O)[C@]12Cc3cn(-c4ccc(F)cc4)nc3C=C1CCN(C(=O)OC(C)(C)C)C2]
and
less than 0.20% of Impurity D:

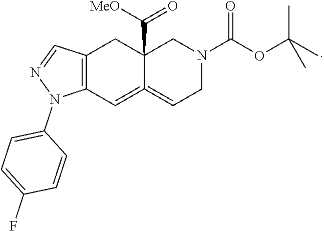

32. The composition of claim 30, wherein the compound of Formula I is prepared by the method of claim 1.
